(19) United States
(12) Patent Application Publication
(10) Pub. No.: US 2012/0183622 A1
(43) Pub. Date: Jul. 19, 2012

(54) ENCAPSULATED CELLS AND COMPOSITES THEREOF

(75) Inventors: Scott A. Guelcher, Franklin, TN (US); Baek-Hee Lee, Seoul (KR); Ruijing Guo, Xuzhou (CN); Bing Li, Troy, NY (US)

(73) Assignee: Vanderbilt University, Nashville, TN (US)

(21) Appl. No.: 13/353,257

(22) Filed: Jan. 18, 2012

Related U.S. Application Data
(60) Provisional application No. 61/433,965, filed on Jan. 18, 2011.

(51) Int. Cl. A61K 35/12; A61K 9/50

(52) U.S. Cl. 424/497; 424/93.7

(57) ABSTRACT

Embodiments of the present invention comprise biodegradable composites including a polyurethane component and cells encapsulated in gel beads, as well as methods of making such composite and uses thereof. In certain embodiments the gel beads are alginate beads. The composites may be moldable and/or injectable. After implantation or injection, a composition may be set to form a porous composite that provides mechanical strength, supports the in-growth of cells, and/or delivers cells to particular tissues. Inventive composites have the advantage of being able to fill irregularly shaped implantation sites, deliver cells in a localized and noninvasive manner, and optimize cell proliferation and differentiation of delivered cells.

### RELATED APPLICATIONS

This application claims priority from U.S. Provisional Application Ser. No. 61/433,965, which was filed on Jan. 18, 2011, the entire disclosure of which is incorporated herein by this reference.

### GOVERNMENT SUPPORT

This invention was made with government support under grant number W81XWH-07-1-0211 from the Orthopadic Extremity Trauma Research Program (DOD) and grant number 1R01AR056138-01A2 from the National Institutes of Health. The United States Government has rights to this invention.

### TECHNICAL FIELD

The present invention generally relates to gel-encapsulated cells and delivery systems thereof. In particular, certain embodiments of the present invention relate to injectable composites comprising polyurethanes and alginate encapsulated cells, wherein the composites deliver cells for tissue repair and regeneration.

### BACKGROUND

Various therapeutic delivery systems have been investigated as treatments for diseases and tissue regeneration [1-9]. Recently, there has been extensive research in the transplantation of living cells into damaged tissues for tissue repair [4-7]. Cell delivery approaches currently under investigation include both direct injection of cells and also delivery of cells within an implanted scaffold [4-7]. However, cells injected directly into the tissue defect can migrate away from the wound and implantation of scaffolds seeded with cells requires invasive surgical techniques [6-9]. In addition, massive cell death induced by the hypoxic and nutrient-limited environment, as well as poor incorporation and integration of the delivered cells, are significant limitations of conventional cell delivery systems [2, 7].

Cell encapsulation in alginate hydrogels represents one of the most widely investigated approaches for cell therapy [10, 11]. Alginate consists of a 3D polymeric network with high water content, which imparts structural and mechanical similarities to macromolecular-based components in natural tissues [11]. The 3D structure of alginate not only facilitates the diffusion of body fluids including nutrients, oxygen and metabolites, but also protects the encapsulated cells against shear forces, chemical reactions, and attack by inflammatory cells [10-14].

Cell-cell contact generally arrests cell growth through contact inhibition [14-17]. For example, relatively large islands of 2D substrates coated with the extracellular matrix (ECM) protein laminin promoted proliferation, while relatively small islands induced apoptosis [15, 17]. Similarly, cell-substrate and cell-cell interactions within 3D gel networks have been suggested to play a critical role in regulating cell proliferation, differentiation, and organ size [18-20]. Encapsulation of cells in alginate beads has been investigated extensively due in part to the flexibility of the process, wherein the physicochemical properties of the gels such as biodegradability, bead size, swelling, gel mesh size, mechanical properties, and cell seeding density can be controlled by varying the chemical composition and processing parameters [11, 21-24]. To promote cell adhesion, proliferation, and differentiation, alginate is frequently modified with cell adhesion peptides and proteins [1, 25-32].

Development of proper cell carriers has also attracted much attention recently. The concepts of material carriers, which will function as synthetic analogs of the extracelluar matrix that provide a substrate for transplanted cell adhesion, control the localization of the cells in vivo, and serve as a template for the formation of new tissue masses from the combination of transplanted cells and interfacing host cells, have been borrowed from tissue engineering field [4]. However, there remains a need for a minimally invasive therapy that effectively deliver cells to a site for tissue repair and regeneration.

### SUMMARY OF THE INVENTION

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned, likewise, those features can be applied to other embodiments of the presently disclosed subject matter, whether listed in this Summary or not. To avoid excessive repetition, this Summary does not list or suggest all possible combinations of such features.

The present inventors have discovered effects of physical microenvironment, including the mesh size and shear modulus of cell-encapsulating gels, on cellular proliferation and differentiation in alginate gels not having modification by peptides. The crosslink density of the gels, which controls the mesh size and shear modulus, may be adjusted by varying the concentrations of the components that form the alginate gels, such as alginic acid and calcium chloride. Mesh size and shear modulus of gel can regulate proliferation and differentiation of cells encapsulated in the alginate beads [37, 38]. To increase cell viability during the encapsulation process, α-minimum essential medium (α-MEM), for example, can be incorporated in alginate beads, which, without being bound be theory or mechanism, enhances the availability of nutrients for encapsulated cells at least during the synthesis process.

Furthermore, polyurethane can be a carrier for cell delivery. However, despite the superiority of polyurethane for cell delivery, cells cannot be encapsulated in it directly. Reactants of polyurethane are highly hydrophobic without nutrients or dissolved oxygen, which is not a friendly environment for cell survivability. Thus a protection barrier is needed during the process of polyurethane scaffold formation. Alginate, because of its abundance, easy gelling properties, and biocompatibility, may encapsulate cells to protect the cells from the attack of the host's immune system [7]. Moreover, cells encapsulated within the calcium alginate hydrogel retain a high level of cell viability [8], and cells may proliferate within an alginate hydrogel for at least 20 days.

Embodiments of the presents invention use injectable polyurethane as a cell carrier for tissue repair, while employing calcium alginate hydrogel as a protection barrier to improve cell survivability.

In order to achieve this goal, it is possible to make alginate beads of a controlled size. Secondly, porosity and inter pore morphology of cured polyurethane scaffolds may be adjusted by, among other things, adjusting the amount of alginate bead loading. Finally, in order to release cells to grow inside polyurethane scaffolds, the alginate hydrogel may be made to degrade relatively quickly after the formation of the scaffold.

Other embodiments of the present invention generally relate to therapeutic delivery systems that are incorporated in scaffolds. These delivery systems can be applied to regenerate damaged tissue. Embodiments of the present invention include an injectable two-component polyurethane (PUR) cell delivery system that immobilizes the cells in an in situ setting scaffold.

Embodiments of the present invention show that cellular outcomes in alginate beads can be regulated by internal pore and mesh structure.

Additionally, embodiments of the present invention can be used to design cell transplantation vehicles that both localize and maintain the viability of the transplanted cells, as well as regulate cell proliferation and differentiation.

In some embodiments of the present invention provide a biodegradable composite comprising a polyurethane component and cells encapsulated in gel beads. The gel beads may have a size of about 200 μm to about 2 mm. Furthermore, certain embodiments of gel beads further comprise a formulation for culturing cells, such as α-MEM, deionized water, PBS, DMEM, or combinations thereof.

In specific embodiments the gel beads are alginate beads. These alginate beads may be made from an alginate solution, for example a sodium alginate solution, comprising 1% to 2% (w/v) alginate. The alginate beads may also be made with a catalyst, including a calcium catalysts such as CaCl₂. The calcium catalyst may be used at any concentration, but in certain embodiments is used at 100 mM to 200 mM concentrations.

Further embodiments of the alginate beads may comprise oxidized alginate, and, for example, 0.1% to 10% of the alginate may be oxidized.

Embodiments of the present invention may comprise 40 wt % to 60 wt % of cells encapsulated in gel beads. The cells may be any cell that is desired to be delivered to a certain site or to particular tissue. In certain instances the cells are delivered for tissue repair and/or regeneration, and may be MC3T3 cell, adipose-derived mesenchymal stem cells, marrow-derived mesenchymal stem cells, any other type of stem cell, any other type of cell that is desired to be delivered, or combinations thereof.

The initial mesh size for embodiments of the gel beads may range from 3 nm to 20 nm. Furthermore, the resulting composites in certain embodiments comprise “blowing-induced pores” having a size of about 10 μm to about 150 μm. These blowing induced pores are formed from, for example, the CO₂ expelled during the synthesis of the composites, and particularly the polymer or polyurethane component. These blowing induced pores may be interconnected. The blowing-induced pores may not be the only pores in a composite. For example, a composite may have pores that form from the presence of gel beads or other chemical or mechanical factors. However, specific embodiments of the present invention have an initial porosity of about 10% to about 50%

Further embodiments of the present invention comprise methods of synthesizing a composite that includes encapsulating cells in gel beads, mixing the cells in gel beads with at least a propolymer and a hardener polyol to form a reactive mixture, and allowing the reactive mixture to react. In specific embodiments the encapsulating is performed by at least mixing cells with an alginate solution to form a cell solution, adding the cell solution to a gelling agent solution through a nozzle, and allowing the gel beads to form. The term “nozzle” as used herein has no limitation other than it refer to an object that substances may pass through. In further embodiments, the size of a resulting gel bead may be modified by adjusting the diameter of the nozzle, adjusting the flow rate of the cell solution passing through the nozzle, and adjusting an applied voltage that is applied to the nozzle.

Furthermore, in embodiments comprising partially oxidized alginate beads, the oxidation may be achieved by reacting a solution including an alginate salt sodium periodate, stopping the reaction with a reaction inhibitor, such as ethylene glycol, precipitating the solution to collect precipitates, and then redissolving the precipitates.

In certain embodiments the hardener polyol may include polyester triol and, optionally, a catalyst. In further specific embodiments, the prepolymer may be a lysine triisocyanate-polyethylene glycol prepolymer.

Embodiments of the present invention including methods of delivering cells to tissue comprising administering to a subject in need thereof an effective amount of a biodegradable composite including a polyurethane component and cells encapsulated in gel beads. The administration of such a composite in specific embodiments regenerates and/or repairs tissue. In specific embodiments the administering an effective amount of the biodegradable composite includes injecting or applying the biodegradable composite on the tissue and allowing the biodegradable composite to cure on the tissue.

### Definitions

The term “alginate” as used herein has the same meaning as that known in the art, and generally refers to any substance comprising alginate. In certain instances, the term alginate may refer to alginic acid, an alginate salt, such as sodium alginate, or both. Those of skill in the art will appreciate the forms of alginate that may be used in instances that call for the use of alginate.

The term “bioactive agent” is used herein to refer to compounds or entities that alter, promote, speed, prolong, inhibit, activate, or otherwise affect biological or chemical events in a subject (e.g., a human). For example, bioactive agents may include, but are not limited to osteogenic, osteoinductive, and osteoconductive agents, anti-HIV substances, anti-cancer substances, antibiotics, immunosuppressants, anti-viral agents, enzyme inhibitors, neurotoxins, opioids, hypnotics, anti-histamines, lubricants, tranquilizers, anti-convulsants, muscle relaxants, anti-Parkinson agents, anti-spasmodics and muscle contractants including channel blockers, miotics and anti-cholinergics, anti-glaucoma compounds, anti-parasite agents, anti-protozoal agents, and/or anti-fungal agents, modulators of cell-extracellular matrix interactions including cell growth inhibitors and anti-adhesion molecules, vasodilating agents, inhibitors of DNA, RNA, or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, anti-depressants, anti-psychotics, targeting agents, chemotactic factors, receptors, neurotransmitters, proteins, cell response modifiers, cells, peptides, polynucleotides, viruses, and vaccines. In certain embodiments, the bioactive agent is a drug. In certain embodiments, the bioactive agent is a small molecule.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, the United States Pharmacopeia-25/National Formulary-20, published by the United States Pharmcopeial Convention, Inc., Rockville Md., 2001, and the “Pharmazeutische Wirkstoffe”, edited by Von Keemann et al., Stuttgart/New York, 1987, all of which are incorporated herein by reference. Drugs for human use listed by the U.S. Food and Drug Administration (FDA) under 21C.F.R. §§330.5, 331 through 361, and 440 through 460, and drugs for veterinary use listed by the FDA under 21C.F.R. §§500 through 589, all of which are incorporated herein by reference, are also considered acceptable for use in accordance with the present invention.

The terms, “biodegradable”, “bioerodable”, or “resorbable” materials, as used herein, are intended to describe materials that degrade under physiological conditions to form a product that can be metabolized or excreted without damage to the subject. In certain embodiments, the product is metabolized or excreted without permanent damage to the subject. Biodegradable materials may be hydrolytically degradable, may require cellular and/or enzymatic action to fully degrade, or both. Biodegradable materials also include materials that are broken down within cells. Degradation may occur by hydrolysis, oxidation, enzymatic processes, phagocytosis, or other processes.

The term “biocompatible” as used herein, is intended to describe materials that, upon administration in vivo, do not induce undesirable side effects. In some embodiments, the material does not induce irreversible, undesirable side effects. In certain embodiments, a material is biocompatible if it does not induce long term undesirable side effects. In certain embodiments, the risks and benefits of administering a material are weighed in order to determine whether a material is sufficiently biocompatible to be administered to a subject.

The term “biomolecules” as used herein, refers to classes of molecules (e.g., proteins, amino acids, peptides, polynucleotides, nucleotides, carbohydrates, sugars, lipids, nucleoproteins, glycoproteins, lipoproteins, steroids, natural products, etc.) that are commonly found or produced in cells, whether the molecules themselves are naturally-occurring or artificially created (e.g., by synthetic or recombinant methods). For example, biomolecules include, but are not limited to, enzymes, receptors, glycosaminoglycans, neurotransmitters, hormones, cytokines, cell response modifiers such as growth factors and chemotactic factors, antibodies, vaccines, haptens, toxins, interferons, ribozymes, anti-sense agents, plasmids, DNA, and RNA. Exemplary growth factors include but are not limited to bone morphogenic proteins (BMP's) and their active fragments or subunits. In some embodiments, the biomolecule is a growth factor, chemotactic factor, cytokine, extracellular matrix molecule, or a fragment or derivative thereof, for example, a cell attachment sequence such as a peptide containing the sequence, RGD.

The term “cells” as used herein has the same meaning as that known in the art. Cell may refer to all types of living or non-living cells from any organism. In certain embodiments, the term cell may also generally refer to a structure that serves as a compartment for other substances.

The term “composite” as used herein, is used to refer to a unified combination of two or more distinct materials. The composite may be homogeneous or heterogeneous. For example, a composite may be a combination of cells encapsulated in gel beads and a polymer; or a combination of encapsulated cells, polymers and a bioactive agent. In certain embodiments, the composite has a particular orientation. The term “scaffold” may also be used herein and, depending on the particular usage, is either synonymous with composite or refers solely to the PUR component of a composite.

The term “effective amount”, as used herein, refers to an amount of the biodegradable composite sufficient to produce a measurable biological response (e.g., tissue regeneration/repair). Actual dosage levels of the biodegradable composite can be varied so as to administer an amount of antioxidant molecules that is effective to achieve the desired response for a particular subject and/or application. The selected dosage level will depend upon a variety of factors including the type of tissue being addressed, the types of cells and gel beads used, combination with other drugs or treatments, severity of the condition being treated, and the physical condition and prior medical history of the subject being treated. Preferably, a minimal dose is administered, and dose is escalated in the absence of dose-limiting toxicity to a minimally effective amount.

The term “encapsulated” as used herein, has at least the well known meaning that the term has in the art. Encapsulation can be defined as packaging a substrate (e.g., solids, chemicals, cells) with another material, such as a gel, including alginate. An encapsulated material may be partially or wholly contained within the encapsulating material. The encapsulated material is held to or within the encapsulating material by any mechanical, chemical, or other force or bond.

The term “flowable polymer material” as used herein, refers to a flowable composition including one or more of monomers, pre-polymers, oligomers, low molecular weight polymers, uncross-linked polymers, partially cross-linked polymers, partially polymerized polymers, polymers, or combinations thereof that have been rendered formable. One skilled in the art will recognize that a flowable polymer material need not be a polymer but may be polymerizable. In some embodiments, flowable polymer materials include polymers that have been heated past their glass transition or melting point. Alternatively or in addition, a flowable polymer material may include partially polymerized polymer, telechelic polymer, or prepolymer. A pre-polymer is a low molecular weight oligomer typically produced through step growth polymerization. The pre-polymer is formed with an excess of one of the components to produce molecules that are all terminated with the same group. For example, a diol and an excess of a diisocyanate may be polymerized to produce isocyanate terminated prepolymer that may be combined with a diol to form a polyurethane. Alternatively or in addition, a flowable polymer material may be a polymer material/solvent mixture that sets when the solvent is removed.

The term “formulation for culturing cell” as used herein, is used to refer to any substance that supports the life and/or growth of cells and/or microorganisms. The term as used herein also has the same meaning as that used in the art. Types of formulations for culturing cells include, but are not limited to, minimum essential mediums, such as α-MEM, phosphate buffer solution (PBS), deionized (DI) water, and Delbecco's Modified Eagle Medium (DMEM).

The term “gel” as used herein, generally refers to a material having a fluidity at room temperature between that of a liquid and solid. A gel may comprise alginate, for instance. There is no limitation on the type of material that may form a gel as long as the fluidity is as described. Furthermore, “gel beads” refers to structures that comprise a gel-like substance. While the gel beads may have a roughly spherical shape, the term bead does not impart any limitation regarding the size or shape of a bead.

The term “porosity” as used herein, refers to the average amount of non-solid space contained in a material (e.g., a composite of the present invention). Such space is considered void of volume even if it contains a substance that is liquid at ambient or physiological temperature, e.g., 0.5° C. to 50° C. Porosity or void volume of a composite can be defined as the ratio of the total volume of the pores (i.e., void volume) in the material to the overall volume of composites. In some embodiments, porosity (ε), defined as the volume fraction pores, can be calculated from composite foam density, which can be measured gravimetrically. Porosity may in certain embodiments refer to “latent porosity” wherein pores are only formed upon diffusion, dissolution, or degradation of a material occupying the pores. In such an instance, pores may be formed after implantation/injection. It will be appreciated by these of ordinary skill in the art that the porosity of a provided composite or composition may change over time, in some embodiments, after implantation/injection (e.g., after leaching of a porogen, when osteoclasts resorbing allograft bone, etc.). For the purpose of the present disclosure, implantation/injection may be considered to be “time zero” (T₀). In some embodiments, the present invention provides composites and/or compositions having a porosity of at least about 30%, at least about 40%, at least about 50%, at least about 60%, at least about 70%, at least about 80%, at least about 90% or more than 90%, at time zero. In certain embodiments, pre-molded composites and/or compositions may have a porosity of at least about 30%, at least about 40%, at least about 50%, at least about 60%, at least about 70%, at least about 80%, at least about 90% or more than 90%, at time zero. In certain embodiments, injectable composites and/or compositions may have a porosity of as low as 3% at time zero. In certain embodiments, injectable composites and/or compositions may cure in situ and have a porosity of at least about 30%, at least about 40%, at least about 50%, at least about 60%, at least about 70%, at least about 80%, at least about 90% or more than 90% after curing.

The term “setting time” as used herein, is approximated by the tack-free time (TFT), which is defined as the time at which the material could be touched with a spatula with no adhesion of the spatula to the foam. At the TFT, the wound could be closed without altering the properties of the material.

The term “subject” as used herein refers to both human and animal subjects. Thus, veterinary therapeutic uses are provided in accordance with the present invention. As such, the present invention provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The term “shaped” as used herein, is intended to characterize a material (e.g., composite) or an osteoimplant refers to a material or osteoimplant of a determined or regular form or configuration in contrast to an indeterminate or vague form or configuration (as in the case of a lump or other solid matrix of special form). Materials may be shaped into any shape, configuration, or size. For example, materials can be shaped as sheets, blocks, plates, disks, cones, pins, screws, tubes, teeth, bones, portions of bones, wedges, cylinders, threaded cylinders, and the like, as well as more complex geometric configurations.

The term “tissue” is used herein to generally refer to an aggregate of cells that perform a particular function or form, at least part of, a particular structure. A particular tissue may comprise one or more types of cells. A non-limiting example of this is brain tissue, which includes neuron and glial cells, capillary endothelial cells, and blood cells. The term also may refer to certain cell lines. Tissue should not be construed as being limited to any particular organism, but may refer to human, animal, or plant tissue, and may even refer to artificial or synthetic tissue.

### DESCRIPTION OF THE DRAWINGS

Illustrative aspects of embodiments of the present invention will be described in detail with reference to the following figures wherein:

FIG. 1 shows (A) size, (B) mass swelling ratio, and (C) calculated mesh size (ξ) of alginate beads measured as a function of immersion time in DI water for up to 10 days; part (D) shows initial mesh size of synthesized alginate beads, where * denotes statistical significance (p<0.05) compared with the 2-200 bead group and all other groups, and ** denotes statistical significance (p<0.05) compared with the 2-100 bead group and 1 wt. % groups;

FIG. 2 shows the elastic moduli of 1-100, 1-200, 2-100, and 2-200 alginate gels measured under shear mode of deformation, where data are presented as average±standard deviation (n=3) and * and ** denote the same statistical significances as in FIG. 1;

FIG. 3 shows optical and fluorescence microscopy images of MC3T3-E1 cells that were encapsulated in 1-100 and 2-200 alginate beads, treated with Live/Dead stain, and cultured in the standard medium for days 1, 4 and 16;

FIG. 4 shows total protein (TP) concentration (mg/ml) measured for 5 alginate beads as a function of culture time under dynamic conditions in standard and osteogenic media;

FIG. 5 shows alkaline phosphatase (ALP) activity (U/mg/min) measured for 5 alginate beads and normalized by total protein concentration as a function of culture time under dynamic conditions in standard and osteogenic media;

FIG. 6 shows osteocalcin secretion by encapsulated MC3T3-E1 cells in alginate beads cultured under dynamic conditions in (A) standard and (B) osteogenic medium for 16 days, where * and ** denote the same statistical significances as in FIG. 1;

FIG. 7 shows data for total protein (TP) and alkaline phosphatase (ALP) activity normalized by total protein (ALP/TP) versus alginate mesh size in standard and osteogenic medium for 1-100, 1-200, 2-100, and 2-200 beads and fit to a straight line by linear regression;

FIG. 8 shows (A) proliferation rate (as measured by d(TP)/dt) versus initial mesh size (ξᵢ) for standard (TP-S) and osteogenic (TP-O) media, and (B) differentiation rate (as measured by d(ALP/TP)/dt) versus initial mesh size (ξᵢ) for TP-S and TP-O media;

FIG. 9 shows the ratio of the dimensionless proliferation:differentiation rates (r_{P/D}) versus (A) initial mesh size (ξᵢ) (filled squares) and dξ/dt (open circles), and (B) alginate gel shear modulus (G), where data were fit to the power law model;

FIG. 10 shows the effect of applied voltage on (A) alginate bead size, and (B) encapsulated cell viability;

FIG. 11 shows SEM images of polyurethane scaffolds incorporating alginate beads (˜500 μm) at (A) 40% loading, (B) 50% loading, and (C) 60% loading;

FIG. 12 shows (A) a comparison of degradation rates between alginate hydrogel and partially oxidized hydrogel, (B)-(D) images depicting degradation of partially oxidized alginate hydrogel inside a polyurethane scaffold at, respectively, days 0, 1, and 4, and (E)-(F) 10× images depicting cell viability in, respectively, 2 mm partially oxidized alginate beads without applied voltage and 1 mm beads with 4 kV applied voltage;

FIG. 13 shows cell viability for >2 mm beads inside polyurethane scaffolds cultured for 5 days (left) and 20 days (right);

FIG. 14 shows optical micrographs of various Ca-alginate/polyurethane (PUR) scaffolds, where (A) and (B) show S21/PUR (alginate bead diameter ˜1 mm) composites, and (C) and (D) show B21/PUR (alginate bead diameter ˜2 mm) composites;

FIG. 15 shows the viability of MC3T3 cell encapsulated in alginate beads incorporated in injectable PUR scaffolds having 50 wt. % and 60 wt. % alginate bead in PUR, where (A) and (B) show data for, respectively, the beads alone and beads incorporated in PUR;

FIG. 16 shows optical and fluorescence microscopes images of cell-alginate microcapsules after soaking for 120 h in CaCl₂/Water (solvent: DI-Water), CaCl₂/α-MEM (solvent: α-MEM), and α-MEM (solvent: α-MEM);

FIG. 17 shows optical and fluorescence microscopes images of injectable PUR scaffolds comprising 50 wt. % and 60 wt. % cell-alginate microcapsulated in alginate bead after soaking for (upper; harvested from PUR scaffold) 48 h and (down; in PUR scaffold) 120 h in α-MEM;

FIG. 18 shows total protein (A, B) and ALP activity/total protein (C, D) in bead/PUR scaffolds as a function of immersion time in α-MEM, where (A) and (C) show CP11 (1 wt. %/100 mM) and CP12 (1 wt. %/100 mM) scaffolds, and (B) and (D) show CP21 (2 wt. %/100 mM) and CP22 (2 wt. %/100 mM) scaffolds;

FIG. 19 shows (A) a photograph, and (B) a schematic of a Var-V1 encapsulation unit used to make certain embodiments of the present invention;

FIG. 20 shows optical microscope images of alginate beads synthesized with various voltages, where the top row from left to right shows beads for 1 kV, 2 kV, and 3 kV, and the bottom row from left to right shows beads for 4 kV, 5 kV, and 6 kV;

FIG. 21 shows a chart of the size of synthesized alginate beads versus voltage;

FIG. 22 shows a chart of cell viability in synthesized alginate beads versus voltage; and

FIG. 23 shows fluorescence microscope images of alginate beads synthesized with various voltages, where the top row from left to right shows beads for 1 kV, 2 kV, and 3 kV, and the bottom row from left to right shows beads for 4 kV, 5 kV, and 6 kV.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding, and no unnecessary limitations are to be understood therefrom.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although many methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a polymer” includes a plurality of such polymers, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations in some embodiments of ±20%, in some embodiments of ±10%, in some embodiments of ±5%, in some embodiments of ±1%, in some embodiments of ±0.5%, and in some embodiments of ±0.1% from the specified amount, as such variations are appropriate to perform the disclosed method. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Alginate consists of a 3D polymeric network with high water content, which imparts structural and mechanical similarities to macromolecular-based components in natural tissues. The 3D structure of alginate allows not only the diffusion of body fluids including nutrients, oxygen and waste, but also the protection of encapsulated cells from external stresses such as mechanical forces and attack by the immune system. Alginate properties such as pore size and swelling can be simply controlled for desired use of a therapeutic delivery system during the synthesis process. Without being bound by theory or mechanism, the present inventors discovered that gel microstructure regulates proliferation and differentiation of cells encapsulated in alginate beads. The present inventors discovered that the reaction conditions of cell-alginate beads significantly control the proliferation and differentiation of the encapsulated cells in vitro. Minimization of damage during cell encapsulation also occurs.

Critical-size tissue defects have a limited capacity for self-healing. Various therapeutic delivery systems, which are incorporated in scaffolds, have been applied to regenerate damaged tissue. Scaffolds incorporating cells (known as cell therapeutic scaffolds) have been widely investigated for diseases and tissue regeneration. Ideally, scaffolds for tissue regeneration undergo controlled degradation to non-cytoxic breakdown products, accelerate cell viability, facilitate transport of nutrients, prevent a hypoxic environment, provide mechanical strength for a desired use, and support integration with the surrounding tissue. Battlefield injuries and fractures often result in penetrating injury and mass loss with various size and shapes.

Embodiments of the present invention comprise composites including a polyurethane (PUR) component and cells encapsulated in gel beads, including alginate beads. Specific embodiments are able to avoid massive cell death induced by the hypoxic environment and transport limitations, as well as poor incorporation and integration. Further embodiments are injectable, and injectability of the scaffold enables handling as a viscous solution and polymerization in situ for various size and shapes. One embodiment of the present invention is an injectable two-component polyurethane (PUR) cell delivery system that immobilizes the cells in an in situ setting scaffold. Without being bound by theory or mechanism, the present inventors discovered that supplement of nutrient and oxygen during polymerization and inter-connectivity of cell-alginate beads and body fluid after polymerization would enhance the viability of encapsulated cells.

Embodiments of the present invention may comprise any type of cell that is desired for a therapy delivery system, and may include stem cells, preosteogenic cells, or any other type of cells.

Certain embodiments of the present invention encapsulate cells in a alginate bead, and the alginate optionally may be oxidized. Oxidation of the alginate, in certain applications, allows the beads to degrade at a faster rate, thereby providing protection to the cells during composite synthesis, but then degrading at a relatively rapid rate to release encapsulated cells.

Certain embodiments of the present invention also comprise composites and method of synthesis that account for the microstructure of gels used to encapsulate cells. For instance, for alginate beads made of alginic acid and a calcium catalyst, such as CaCl₂, increasing the concentration of the calcium catalyst and/or alginic acid increases the extent to which cell differentiation occurs within a composite. On the other hand, decreasing calcium catalyst and/or alginic acid concentrations may decrease the proliferation rate of cells within a composite.

In certain embodiments, the present inventors have discovered that high density gels having a small initial mesh size and a more rigid structures promote differentiation, whereas low density, larger initial mesh size, and more complex structures promote proliferation. By accounting for these behaviors, one of ordinary skill in the art can modify the substances used to form an encapsulated cell so as to optimize differentiation and proliferation of those cells.

Still further embodiments of the present invention comprise cells encapsulated in gel beads that include a formulation for cell culture, such as α-MEM. Formulations for cell culture incorporated into the gel beads may provide nutrients and other essential substances to cells potentially both while the cells are encapsulated and after the bead itself has degraded.

In specific embodiments of the present invention, cells (e.g., MC3T3-E1 osteoprogenitor cells) are encapsulated in alginate beads with varying mesh size and shear modulus, which effects the microstructure of the gel and cell proliferation and differentiation. By varying the concentration of the alginate and CaCl₂ precursor solutions, gels with initial mesh sizes ranging from 4-20 nm can be synthesized.

In further embodiments of the present invention, encapsulation of the cells using alginate and CaCl₂ solutions prepared from formulations for cell culture, such as α-MEM or the like, increased the initial cell viability to, for example, >98% from the value of 60% observed when the precursor solutions were prepared from DI water. Without being bound be theory or mechanism, the increase in cell viability was attributed to the availability of nutrients provided by α-MEM during the entire encapsulation process.

In embodiments of the present invention, the initial mesh sizes decrease with increasing concentration of alginic acid and CaCl₂.

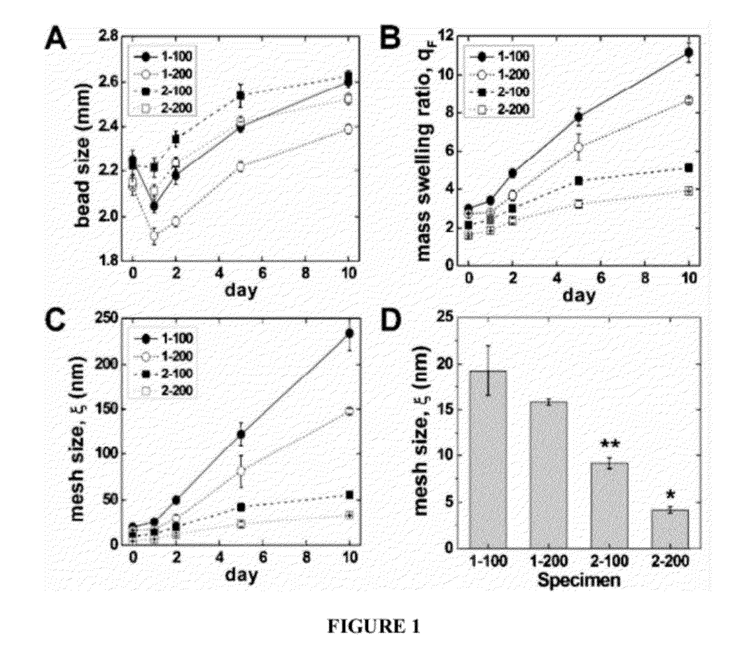

Embodiments of the present invention takes into account that cells (˜10-20 μm) are 3 orders of magnitude larger than initial mesh sizes (e.g., 3-20 nm) of embodied networks, and cross-linking of the alginate around cells may result in micron-size defects in which the cells are embedded. When incubated in buffer or water (e.g., Ca²⁺-free medium), diffusion of the divalent ions (e.g., Ca²⁺) ionically cross-linking the embodied gel into the surrounding medium can result in dissolution of the gel, which is consistent with the data in FIGS. 1A and B showing an increase in bead size and swelling when incubated in α-MEM for up to 10 days. Swelling of alginate gels can be associated with changes in chain stretching and conformation [49, 50], which contribute to the increase in mesh size (FIG. 1C). As the crosslinks degrade, micron-size defects resulting from the presence of the cells may grow in size, thereby creating additional space in which cells can proliferate. Consequently, cell proliferation may increase with increasing rate of crosslink degradation (approximated by dξ/dt). See FIG. 9A.

To further show the properties of the alginate gels regulate cell fate, the dimensionless ratio of proliferation to differentiation (r_{P/D}, Eq (8)) is plotted versus initial mesh size, rate of increase in mesh size (dξ/dt), and shear modulus G in FIG. 9. The value of r_{P/D} scales with ξ^{5.7} and G^{-3.2}, which is in reasonable agreement with the scaling of G with ξ^{-1/2} predicted by Eqs (5) and (6). Since the molecular weight between crosslinks determines both the mesh size and shear modulus, it is difficult to separate the effects of these two parameters on proliferation and differentiation of cells in crosslinked polymers. The dramatic difference in scaling of the proliferation rate with modulus between 2D and 3D suggests that when cells are confined in a 3D gel, the mesh size may have a predominant role in regulating cell fate relative to the rigidity of the matrix.

In embodiments of the present invention, microstructure and mechanical properties influence the behavior of cells encapsulated in an alginate gel. In specific embodiments using MC3T3-E1 pre-osteoblast cells, cells were used as a model system to show that the microstructure of the 3D gel regulates proliferation and differentiation of encapsulated cells. Furthermore, certain embodiments present a potential solution to reducing host immune reaction by delivering stem cells harvested from the patient.

Compliant gels with larger mesh size supported cell proliferation, while rigid gels with smaller mesh size enhanced expression of markers of osteoblast differentiation, which suggests that cellular outcomes in 3D alginate beads are regulated by the nanostructure of the networks. This approach is useful for the design of cell transplantation vehicles that both localize and maintain the viability of the transplanted cells, as well as regulate cell proliferation and differentiation.

Furthermore, embodiments of the present invention also comprises injectable polyurethane scaffolds employed as carriers of cell therapy for tissue repair. To protect cells during the formation of polyurethane, certain embodied utilize calcium alginate hydrogel as a protection barrier containing cell culture medium inside the bead structure. An electronic bead maker can be used to control alginate bead size, and high cell viability may be maintained even with the treatment of the applied voltage (e.g., up to 6 kV). In order to encapsulate cells homogeneously in polyurethane, as well as to keep high cell viability, bead diameter of 300 μm (4.4 kV) was selected.

Embodiments of the present invention may be loaded with any desired amount of encapsulated cells. For example, embodiments of the present invention comprise 10 wt %, 20 wt %, 30 wt %, 40 wt %, 50 wt %, 60 wt %, 70 wt %, 80 wt %, and 90 wt % of encapsulated cells. Specific embodiments of the present invention comprise about 40 wt % to about 60 wt % of encapsulated cells. Any amount of cells may be added to a composite, although cell loading may affect the structural integrity and characteristics of a composite.

Certain embodiments of the present invention have also been designed so that the encapsulation around cells degrades at a faster rate, and may degrade in, for example, 1 to 2 days. Certain embodiments utilize unmodified alginate beads to encapsulate cells, but these beads may take more than two days to degrade. On the other hand, specific embodiments utilize partially or fully oxidized alginate to form the beads to encapsulate cells. Certain embodiments comprise alginate that is about 5% oxidized. Certain embodiments comprising oxidized alginate have the oxidized alginate degrade at a faster rate than unmodified alginate when implanted in or on a subject. Faster degradation allow cells to be released from encapsulation at a earlier point in time following administration, which may accelerate a composite's ability to deliver cells to a site for tissue repair and regeneration. However, partially oxidized alginate beads do not degrade for several hours or even a couple of days. Therefore, partially oxidized alginate beads have the advantage of being able to protect cells during the mixing and curing of the polymer component of a biodegradable composite, yet degrade at a rate that allows for relatively rapid release of cells and subsequent tissue repair and regeneration.

Embodiments of the present invention comprise encapsulated cells that are of various dimensions. The encapsulated cells may be in the form a bead, for instance an alginate bead, that is anywhere from 200 μm to 2 mm in diameter. In certain embodiments, 200 μm to 300 μm beads are desired because they create pores that readily allow for cell integration and incorporation. However, other embodiments utilize beads that range from 300 μm to 800 μm, and other embodiments may comprise beads that are as large as 2 mm. The bead size will vary depending on the tissue to be repair and the application method. For instance, it is possible that larger beads (e.g., 2 mm) may be difficult to inject, depending on the diameter of the needle used for injection. For example, beads may be 200 μm, 300 μm, 400 μm, 500 μm, 600 μm, 700 μm, 800 μm, 900 μm, 1.0 mm, 1.1 mm, 1.2 mm, 1.3 mm, 1.4 mm, 1.5 mm, 1.6 mm, 1.7 mm, 1.8 mm, 1.9 mm, or 2.0 mm in size.

Certain embodiments of present invention are able to achieve superior and unexpected results, including their ability to cure in situ and release cells for therapy, because they are formed from a biodegradable polyurethane component. The following describes the polymer component in more detail, including its synthesis and other components that may be delivered with the PUR.

Polymer Component

Synthetic polymers can be designed with properties targeted for a given clinical application. According to the present invention, polyurethanes (PUR) are a useful class of biomaterials due to the fact that they can be injectable or moldable as a reactive liquid that subsequently cures to form a porous composite These materials also have tunable degradation rates, which are shown to be highly dependent on the choice of polyol and isocyanate components (Hafeman et al., Pharmaceutical Research 2008; 25(10):2387-99; Storey et al., J Poly Sci Pt A: Poly Chem 1994; 32:2345-63; Skarja et al., J App Poly Sci 2000; 75:1522-34). Polyurethanes have tunable mechanical properties, which can also be enhanced with the addition of bone particles and/or other components (Adhikari et al., Biomaterials 2008; 29:3762-70; Goma et al., J Biomed Mater Res Pt A 2003; 67A(3):813-27) and exhibit elastomeric rather than brittle mechanical properties. Thus, for certain embodiments of the present invention, the polymer component may be a “polyurethane component”.

Polyurethanes can be made by reacting together the components of a two-component composition, one of which includes a polyisocyanate while the other includes a component having two or more hydroxyl groups (i.e., polyols) to react with the polyisocyanate. For example, U.S. Pat. No. 6,306,177, discloses a method for repairing a tissue site using polyurethanes, the content of which is incorporated by reference.

It is to be understood that by “a two-component composition” it means a composition comprising two essential types of polymer components. In some embodiments, such a composition may additionally comprise one or more other optional components.

In some embodiments, polyurethane is a polymer that has been rendered formable through combination of two liquid components (i.e., a polyisocyanate prepolymer and a polyol). In some embodiments, a polyisocyanate prepolymer or a polyol may be a molecule with two or three isocyanate or hydroxyl groups respectively. In some embodiments, a polyisocyanate prepolymer or a polyol may have at least four isocyanate or hydroxyl groups respectively.

Synthesis of porous polyurethane results from a balance of two simultaneous reactions. Reactions, in some embodiments, are illustrated below in Scheme 1. One is a gelling reaction, where an isocyanates and a polyester polyol react to form urethane bonds. The one is a blowing reaction. An isocyanate can react with water to form carbon dioxide gas, which acts as a lowing agent to form pores of polyurethane foam. The relative rates of these reactions determine the scaffold morphology, working time, and setting time.

Exemplary gelling and blowing reactions in forming of polyurethane are shown in Scheme 1 below, where R₁, R₂ and R₃, for example, can be oligomers of caprolactone, lactide and glycolide respectively.

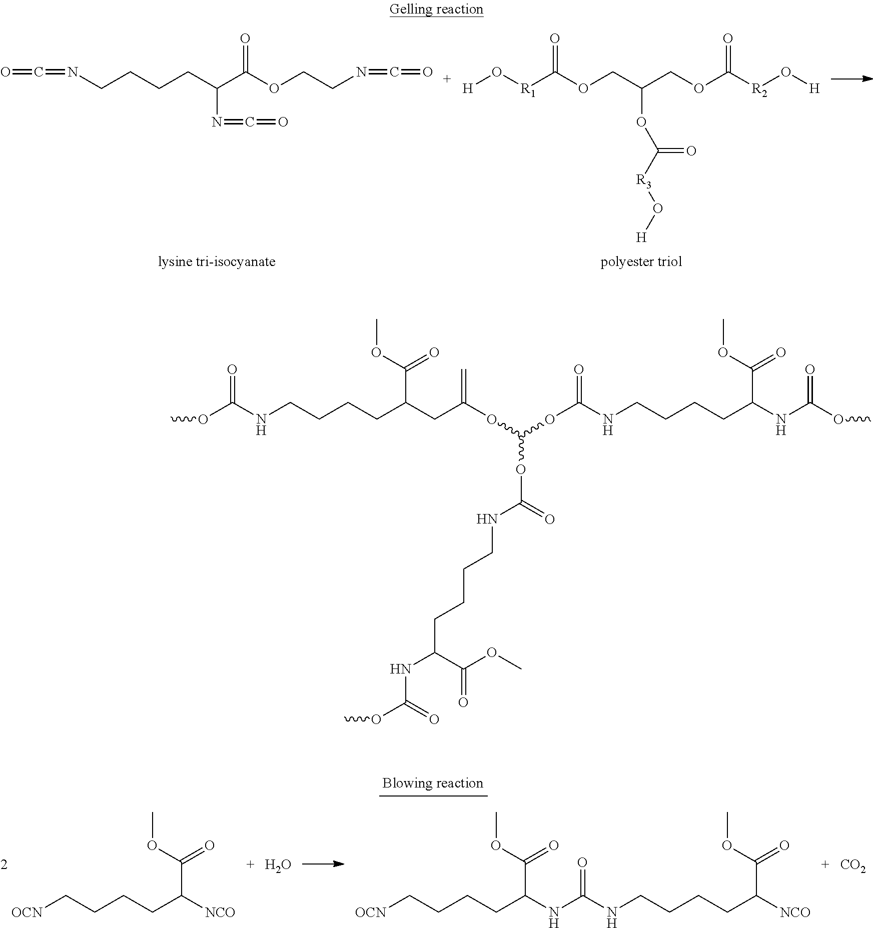

Biodegradable polyurethane scaffolds synthesized from aliphatic polyisocyanates been shown to degrade into non-toxic compounds and support cell attachment and proliferation in vitro. A variety of polyurethane polymers suitable for use in the present invention are known in the art, many of which are listed in commonly owned applications: U.S. Ser. No. 10/759,904 filed on Jan. 16, 2004, entitled “Biodegradable polyurethanes and use thereof” and published under No. 2005-0013793; U.S. Ser. No. 11/667,090 filed on Nov. 5, 2005, entitled “Degradable polyurethane foams” and published under No. 2007-0299151; U.S. Ser. No. 12/298,158 filed on Apr. 24, 2006, entitled “Biodegradable polyurethanes” and published under No. 2009-0221784; all of which are incorporated herein by reference. Polyurethanes described in U.S. Ser. No. 11/336,127 filed on Jan. 19, 2006 and published under No. 2006-0216323, which is entitled “Polyurethanes for Osteoimplants” and incorporated herein by reference, may be used in some embodiments of the present invention.

Polyurethanes foams may be prepared by contacting an isocyanate-terminated prepolymer (component 1, e.g, polyisocyanate prepolymer) with a hardener (component 2) that includes at least a polyol (e.g., a polyester polyol) and water, a catalyst and optionally, a stabilizer, a porogen, PEG, etc. In some embodiments, multiple polyurethanes (e.g., different structures, difference molecular weights) may be used in a composite/composition of the present invention. In some embodiments, other biocompatible and/or biodegradable polymers may be used with polyurethanes in accordance with the present invention. In some embodiments, biocompatible co-polymers and/or polymer blends of any combination thereof may be exploited.

Polyurethanes used in accordance with the present invention can be adjusted to produce polymers having various physiochemical properties and morphologies including, for example, flexible foams, rigid foams, elastomers, coatings, adhesives, and sealants. The properties of polyurethanes are controlled by choice of the raw materials and their relative concentrations. For example, thermoplastic elastomers are characterized by a low degree of cross-linking and are typically segmented polymers, consisting of alternating hard (diisocyanates and chain extenders) and soft (polyols) segments. Thermoplastic elastomers are formed from the reaction of diisocyanates with long-chain diols and short-chain diol or diamine chain extenders. In some embodiments, pores in bone/polyurethanes composites in the present invention are interconnected and have a diameter ranging from approximately 50 to approximately 1000 microns.

Prepolymer. Polyurethane prepolymers can be prepared by contacting a polyol with an excess (typically a large excess) of a polyisocyanate. The resulting prepolymer intermediate includes an adduct of polyisocyanates and polyols solubilized in an excess of polyisocyanates. Prepolymer can, in some embodiments, be formed by using an approximately stoichiometric amount of polyisocyanates in forming a prepolymer and subsequently adding additional polyisocyanates. The prepolymer therefore exhibits both low viscosity, which facilitates processing, and improved miscibility as a result of the polyisocyanate-polyol adduct. Polyurethane networks can, for example, then be prepared by reactive liquid molding, wherein the prepolymer is contacted with a polyester polyol to form a reactive liquid mixture (i.e., a two-component composition) which is then cast into a mold and cured.

Polyisocyanates or multi-isocyanate compounds for use in the present invention include aliphatic polyisocyanates. Exemplary aliphatic polyisocyanates include, but are not limited to, lysine diisocyanate, an alkyl ester of lysine diisocyanate (for example, the methyl ester or the ethyl ester), lysine triisocyanate, hexamethylene diisocyanate, isophorone diisocyanate (IPDI), 4,4′-dicyclohexylmethane diisocyanate (H₁₂MDI), cyclohexyl diisocyanate, 2,2,4-(2,2,4)-trimethylhexamethylene diisocyanate (TMDI), dimers prepared form aliphatic polyisocyanates, trimers prepared from aliphatic polyisocyanates and/or mixtures thereof. In some embodiments, hexamethylene diisocyanate (HDI) trimer sold as Desmodur N3300A may be a polyisocyanate utilized in the present invention. In some embodiments, polyisocyanates used in the present invention includes approximately 10 to 55% NCO by weight (wt % NCO=100*(42/Mw)). In some embodiments, polyisocyanates include approximately 15 to 50% NCO.

Polyisocyanate prepolymers provide an additional degree of control over the structure of biodegradable polyurethanes. Prepared by reacting polyols with isocyanates, NCO-terminated prepolymers are oligomeric intermediates with isocyanate functionality as shown in Scheme 1. To increase reaction rates, urethane catalysts (e.g., tertiary amines) and/or elevated temperatures (60-90° C.) may be used (see, Guelcher, Tissue Engineering: Part B, 14 (1) 2008, pp 3-17).

Polyols used to react with polyisocyanates in preparation of NCO-terminated prepolymers refer to molecules having at least two functional groups to react with isocyanate groups. In some embodiments, polyols have a molecular weight of no more than 1000 g/mol. In some embodiments, polyols have a rang of molecular weight between about 100 g/mol to about 500 g/mol. In some embodiments, polyols have a rang of molecular weight between about 200 g/mol to about 400 g/mol. In certain embodiments, polyols (e.g., PEG) have a molecular weight of about 200 g/mol. Exemplary polyols include, but are not limited to, PEG, glycerol, pentaerythritol, dipentaerythritol, tripentaerythritol, 1,2,4-butanetriol, trimethylolpropane, 1,2,3-trihydroxyhexane, myo-inositol, ascorbic acid, a saccharide, or sugar alcohols (e.g., mannitol, xylitol, sorbitol etc.). In some embodiments, polyols may comprise multiple chemical entities having reactive hydrogen functional groups (e.g., hydroxy groups, primary amine groups and/or secondary amine groups) to react with the isocyanate functionality of polyisocyanates.

In some embodiments, polyisocyanate prepolymers are resorbable. Zhang and coworkers synthesized biodegradable lysine diisocyanate ethyl ester (LDI)/glucose polyurethane foams proposed for tissue engineering applications. In those studies, NCO-terminated prepolymers were prepared from LDI and glucose. The prepolymers were chain-extended with water to yield biocompatible foams which supported the growth of rabbit bone marrow stromal cells in vitro and were non-immunogenic in vivo. (see Zhang, et al., Biomaterials 21: 1247-1258 (2000), and Zhang, et al., Tiss. Eng., 8(5): 771-785 (2002), both of which are incorporated herein by reference).

In some embodiments, prepared polyisocyanate prepolymer can be a flowable liquid at processing conditions. In general, the processing temperature is no greater than 60° C. In some embodiments, the processing temperature is ambient temperature (25° C.).

Polyols. Polyols utilized in accordance with the present invention can be amine- and/or hydroxyl-terminated compounds and include, but are not limited to, polyether polyols (such as polyethylene glycol (PEG or PEO), polytetramethylene etherglycol (PTMEG), polypropylene oxide glycol (PPO)); amine-terminated polyethers; polyester polyols (such as polybutylene adipate, caprolactone polyesters, castor oil); and polycarbonates (such as poly(1,6-hexanediol) carbonate). In some embodiments, polyols may be (1) molecules having multiple hydroxyl or amine functionality, such as glucose, polysaccharides, and castor oil; and (2) molecules (such as fatty acids, triglycerides, and phospholipids) that have been hydroxylated by known chemical synthesis techniques to yield polyols.

Polyols used in the present invention may be polyester polyols. In some embodiments, polyester polyols may include polyalkylene glycol esters or polyesters prepared from cyclic esters. In some embodiments, polyester polyols may include poly(ethylene adipate), poly(ethylene glutarate), poly(ethylene azelate), poly(trimethylene glutarate), poly(pentamethylene glutarate), poly(diethylene glutarate), poly(diethylene adipate), poly(triethylene adipate), poly(1,2-propylene adipate), mixtures thereof, and/or copolymers thereof. In some embodiments, polyester polyols can include, polyesters prepared from caprolactone, glycolide, D, L-lactide, mixtures thereof, and/or copolymers thereof. In some embodiments, polyester polyols can, for example, include polyesters prepared from castor-oil. When polyurethanes degrade, their degradation products can be the polyols from which they were prepared from.

In some embodiments, polyester polyols can be miscible with prepared prepolymers used in reactive liquid mixtures (i.e., two-component composition) of the present invention. In some embodiments, surfactants or other additives may be included in the reactive liquid mixtures to help homogenous mixing.

The glass transition temperature (Tg) of polyester polyols used in the reactive liquids to form polyurethanes can be less than 60° C., less than 37° C. (approximately human body temperature) or even less than 25° C. In addition to affecting flowability at processing conditions, Tg can also affect degradation. In general, a Tg of greater than approximately 37° C. will result in slower degradation within the body, while a Tg below approximately 37° C. will result in faster degradation.

Molecular weight of polyester polyols used in the reactive liquids to form polyurethanes can, for example, be adjusted to control the mechanical properties of polyurethanes utilized in accordance with the present invention. In that regard, using polyester polyols of higher molecular weight results in greater compliance or elasticity. In some embodiments, polyester polyols used in the reactive liquids may have a molecular weight less than approximately 3000 Da. In certain embodiments, the molecular weight may be in the range of approximately 200 to 2500 Da or 300 to 2000 Da. In some embodiments, the molecular weight may be approximately in the range of approximately 450 to 1800 Da or 450 to 1200 Da.

In some embodiments, a polyester polyol comprise poly(caprolactone-co-lactide-co-glycolide), which has a molecular weight in a range of 200 Da to 2500 Da, or 300 Da to 2000 Da.

In some embodiments, polyols may include multiply types of polyols with different structures, molecular weight, properties, etc.

Additional Components. In accordance with the present invention, two-component compositions (i.e., polyprepolymers and polyols) to form porous composites may be used with other agents and/or catalysts. Zhang et al. have found that water may be an adequate blowing agent for a lysine diisocyanate/PEG/glycerol polyurethane (see Zhang, et al., Tissue Eng. 2003 (6):1143-57) and may also be used to form porous structures in polyurethanes. Other blowing agents include dry ice or other agents that release carbon dioxide or other gases into the composite. Alternatively, or in addition, porogens (see detail discussion below) such as salts may be mixed in with reagents and then dissolved after polymerization to leave behind small voids.

Two-component compositions and/or the prepared composites used in the present invention may include one or more additional components. In some embodiments, inventive compositions and/or composites may includes, water, a catalyst (e.g., gelling catalyst, blowing catalyst, etc.), a stabilizer, a plasticizer, a porogen, a chain extender (for making of polyurethanes), a pore opener (such as calcium stearate, to control pore morphology), a wetting or lubricating agent, etc. (See, U.S. Ser. No. 10/759,904 published under No. 2005-0013793, and U.S. Ser. No. 11/625,119 published under No. 2007-0191963; both of which are incorporated herein by reference).

In some embodiments, inventive compositions and/or composites may include and/or be combined with encapsulated cells (e.g., stem cell encapsulated in alginate beads). For example, when composites used in tissue healing, solid fillers including cells can help deliver cells to a particular site with limited cell migration and death.

In certain embodiments, additional biocompatible polymers (e.g., PEG) or co-polymers can be used with compositions and composites in the present invention.

Water. Water may be a blowing agent to generate porous polyurethane-based composites. Porosity of bone/polymer composites increased with increasing water content, and biodegradation rate accelerated with decreasing polyester half-life, thereby yielding a family of materials with tunable properties that are usefull in the present invention. See, Guelcher et al., Tissue Engineering, 13(9), 2007, pp 2321-2333, which is incorporated by reference.

In some embodiments, an amount of water is about 0.5, 1, 1.5, 2, 3, 4 5, 6, 7, 8, 9, 10 parts per hundred parts (pphp) polyol. In some embodiments, water has an approximate rang of any of such amounts.

Catalyst. In some embodiments, at least one catalyst is added to form reactive liquid mixture (i.e., two-component compositions). A catalyst, for example, can be non-toxic (in a concentration that may remain in the polymer).

A catalyst can, for example, be present in two-component compositions in a concentration in the range of approximately 0.5 to 5 parts per hundred parts polyol (pphp) and, for example, in the range of approximately 0.5 to 2, or 2 to 3 pphp. A catalyst can, for example, be an amine compound. In some embodiments, catalyst may be an organometallic compound or a tertiary amine compound. In some embodiments the catalyst may be stannous octoate (an organobismuth compound), triethylene diamine, bis(dimethylaminoethyl)ether, dimethylethanolamine, dibutyltin dilaurate, and Coscat organometallic catalysts manufactured by Vertullus (a bismuth based catalyst), or any combination thereof.

Stabilizer. In some embodiments, a stabilizer is nontoxic (in a concentration remaining in the polyurethane foam) and can include a non-ionic surfactant, an anionic surfactant or combinations thereof. For example, a stabilizer can be a polyethersiloxane, a salt of a fatty sulfonic acid or a salt of a fatty acid. In certain embodiments, a stabilizer is a polyethersiloxane, and the concentration of polyethersiloxane in a reactive liquid mixture can, for example, be in the range of approximately 0.25 to 4 parts per hundred polyol. In some embodiments, polyethersiloxane stabilizer are hydrolyzable.

In some embodiments, the stabilizer can be a salt of a fatty sulfonic acid. Concentration of a salt of the fatty sulfonic acid in a reactive liquid mixture can be in the range of approximately 0.5 to 5 parts per hundred polyol. Examples of suitable stabilizers include a sulfated castor oil or sodium ricinoleicsulfonate.

Stabilizers can be added to a reactive liquid mixture of the present invention to, for example, disperse prepolymers, polyols and other additional components, stabilize the rising carbon dioxide bubbles, and/or control pore sizes of inventive composites. Although there has been a great deal of study of stabilizers, the operation of stabilizers during foaming is not completely understood. Without limitation to any mechanism of operation, it is believed that stabilizers preserve the thermodynamically unstable state of a polyurethane foam during the time of rising by surface forces until the foam is hardened. In that regard, foam stabilizers lower the surface tension of the mixture of starting materials and operate as emulsifiers for the system. Stabilizers, catalysts and other polyurethane reaction components are discussed, for example, in Oertel, Günter, ed., Polyurethane Handbook, Hanser Gardner Publications, Inc. Cincinnati, Ohio, 99-108 (1994). A specific effect of stabilizers is believed to be the formation of surfactant mono layers at the interface of higher viscosity of bulk phase, thereby increasing the elasticity of surface and stabilizing expanding foam bubbles.

Chain extender. To prepare high-molecular-weight polymers, prepolymers are chain extended by adding a short-chain (e.g., <500 g/mol) polyamine or polyol. In certain embodiments, water may act as a chain extender. In some embodiments, addition of chain extenders with a functionality of two (e.g., diols and diamines) yields linear alternating block copolymers.

Plasticizer. In some embodiments, inventive compositions and/or composites include one or more plasticizers. Plasticizers are typically compounds added to polymers or plastics to soften them or make them more pliable. According to the present invention, plasticizers soften, make workable, or otherwise improve the handling properties of polymers or composites. Plasticizers also allow inventive composites to be moldable at a lower temperature, thereby avoiding heat induced tissue necrosis during implantation. Plasticizer may evaporate or otherwise diffuse out of the composite over time, thereby allowing composites to harden or set. Without being bound to any theory, plasticizer are thought to work by embedding themselves between the chains of polymers. This forces polymer chains apart and thus lowers the glass transition temperature of polymers. In general, the more plasticizer added, the more flexible the resulting polymers or composites will be.

In some embodiments, plasticizers are based on an ester of a polycarboxylic acid with linear or branched aliphatic alcohols of moderate chain length. For example, some plasticizers are adipate-based. Examples of adipate-based plasticizers include bis(2-ethylhexyl)adipate (DOA), dimethyl adipate (DMAD), monomethyl adipate (MMAD), and dioctyl adipate (DOA). Other plasticizers are based on maleates, sebacates, or citrates such as bibutyl maleate (DBM), diisobutylmaleate (DIBM), dibutyl sebacate (DBS), triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC), and trimethylcitrate (TMC). Other plasticizers are phthalate based. Examples of phthalate-based plasticizers are N-methyl phthalate, bis(2-ethylhexyl) phthalate (DEHP), diisononyl phthalate (DINP), bis(n-butyl)phthalate (DBP), butyl benzyl phthalate (BBzP), diisodecyl phthalate (DOP), diethyl phthalate (DEP), diisobutyl phthalate (DIBP), and di-n-hexyl phthalate. Other suitable plasticizers include liquid polyhydroxy compounds such as glycerol, polyethylene glycol (PEG), triethylene glycol, sorbitol, monacetin, diacetin, and mixtures thereof. Other plasticizers include trimellitates (e.g., trimethyl trimellitate (TMTM), tri-(2-ethylhexyl) trimellitate (TEHTM-MG), tri-(n-octyl,n-decyl) trimellitate (ATM), tri-(heptyl,nonyl) trimellitate (LTM), n-octyl trimellitate (OTM)), benzoates, epoxidized vegetable oils, sulfonamides (e.g., N-ethyl toluene sulfonamide (ETSA), N-(2-hydroxypropyl)benzene sulfonamide (HP BSA), N-(n-butyl) butyl sulfonamide (BBSA-NBBS)), organophosphates (e.g., tricresyl phosphate (TCP), tributyl phosphate (TBP)), glycols/polyethers (e.g., triethylene glycol dihexanoate, tetraethylene glycol diheptanoate), and polymeric plasticizers. Other plasticizers are described in Handbook of Plasticizers (G. Wypych, Ed., ChemTec Publishing, 2004), which is incorporated herein by reference. In certain embodiments, other polymers are added to the composite as plasticizers. In certain particular embodiments, polymers with the same chemical structure as those used in the composite are used but with lower molecular weights to soften the overall composite. In other embodiments, different polymers with lower melting points and/or lower viscosities than those of the polymer component of the composite are used.

In some embodiments, polymers used as plasticizer are poly(ethylene glycol) (PEG). PEG used as a plasticizer is typically a low molecular weight PEG such as those having an average molecular weight of 1000 to 10000 g/mol, for example, from 4000 to 8000 g/mol. In certain embodiments, PEG 4000, PEG 5000, PEG 6000, PEG 7000, PEG 8000 or combinations thereof are used in inventive composites. For example, plasticizer (PEG) is useful in making more moldable composites that include poly(lactide), poly(D,L-lactide), poly(lactide-co-glycolide), poly(D,L-lactide-co-glycolide), or poly(caprolactone). Plasticizer may comprise 1-40% of inventive composites by weight. In some embodiments, the plasticizer is 10-30% by weight. In some embodiments, the plasticizer is approximately 10%, 15%, 20%, 25%, 30% or 40% by weight. In other embodiments, a plasticizer is not used in the composite. For example, in some polycaprolactone-containing composites, a plasticizer is not used.

In some embodiments, inert plasticizers may be used. In some embodiments, a plasticizer may not be used in the present invention.

Porogen. Porosity of inventive composites may be accomplished using any means known in the art. Exemplary methods of creating porosity in a composite include, but are not limited to, particular leaching processes, gas foaming processing, supercritical carbon dioxide processing, sintering, phase transformation, freeze-drying, cross-linking, molding, porogen melting, polymerization, melt-blowing, and salt fusion (Murphy et al., Tissue Engineering 8(1):43-52, 2002; incorporated herein by reference). For a review, see Karageorgiou et al., Biomaterials 26:5474-5491, 2005; incorporated herein by reference. Porosity may be a feature of inventive composites during manufacture or before implantation, or porosity may only be available after implantation. For example, a implanted composite may include latent pores. These latent pores may arise from including porogens in the composite.

Porogens may be any chemical compound that will reserve a space within the composite while the composite is being molded and will diffuse, dissolve, and/or degrade prior to or after implantation or injection leaving a pore in the composite. Porogens may have the property of not being appreciably changed in shape and/or size during the procedure to make the composite moldable. For example, a porogen should retain its shape during the heating of the composite to make it moldable. Therefore, a porogen does not melt upon heating of the composite to make it moldable. In certain embodiments, a porogen has a melting point greater than about 60° C., greater than about 70° C., greater than about 80° C., greater than about 85° C., or greater than about 90° C.

Porogens may be of any shape or size. A porogen may be spheroidal, cuboidal, rectangular, elonganted, tubular, fibrous, disc-shaped, platelet-shaped, polygonal, etc. In certain embodiments, the porogen is granular with a diameter ranging from approximately 100 microns to approximately 800 microns. In certain embodiments, a porogen is elongated, tubular, or fibrous. Such porogens provide increased connectivity of pores of inventive composite and/or also allow for a lesser percentage of the porogen in the composite.

Amount of porogens may vary in inventive composite from 1% to 80% by weight. In certain embodiments, the plasticizer makes up from about 5% to about 80% by weight of the composite. In certain embodiments, a plasticizer makes up from about 10% to about 50% by weight of the composite. Pores in inventive composites are thought to improve the osteoinductivity or osteoconductivity of the composite by providing holes for cells such as osteoblasts, osteoclasts, fibroblasts, cells of the osteoblast lineage, stem cells, etc. Pores provide inventive composites with biological in growth capacity. Pores may also provide for easier degradation of inventive composites as bone is formed and/or remodeled. In some embodiments, a porogen is biocompatible.

A porogen may be a gas, liquid, or solid. Exemplary gases that may act as porogens include carbon dioxide, nitrogen, argon, or air. Exemplary liquids include water, organic solvents, or biological fluids (e.g., blood, lymph, plasma). Gaseous or liquid porogen may diffuse out of the osteoimplant before or after implantation thereby providing pores for biological in-growth. Solid porogens may be crystalline or amorphous. Examples of possible solid porogens include water soluble compounds. Exemplary porogens include carbohydrates (e.g., sorbitol, dextran (poly(dextrose)), starch), salts, sugar alcohols, natural polymers, synthetic polymers, and small molecules.

In some embodiments, carbohydrates are used as porogens in inventive composites. A carbohydrate may be a monosaccharide, disaccharide, or polysaccharide. The carbohydrate may be a natural or synthetic carbohydrate. In some embodiments, the carbohydrate is a biocompatible, biodegradable carbohydrate. In certain embodiments, the carbohydrate is a polysaccharide. Exemplary polysaccharides include cellulose, starch, amylose, dextran, poly(dextrose), glycogen, etc.

In certain embodiments, a polysaccharide is dextran. Very high molecular weight dextran has been found particularly useful as a porogen. For example, the molecular weight of the dextran may range from about 500,000 g/mol to about 10,000,000 g/mol, preferably from about 1,000,000 g/mol to about 3,000,000 g/mol. In certain embodiments, the dextran has a molecular weight of approximately 2,000,000 g/mol. Dextrans with a molecular weight higher than 10,000,000 g/mol may also be used as porogens. Dextran may be used in any form (e.g., particles, granules, fibers, elongated fibers) as a porogen. In certain embodiments, fibers or elongated fibers of dextran are used as a porogen in inventive composites. Fibers of dextran may be formed using any known method including extrusion and precipitation. Fibers may be prepared by precipitation by adding an aqueous solution of dextran (e.g., 5-25% dextran) to a less polar solvent such as a 90-100% alcohol (e.g., ethanol) solution. The dextran precipitates out in fibers that are particularly useful as porogens in the inventive composite. Once the composite with dextran as a porogen is implanted into a subject, the dextran dissolves away very quickly. Within approximately 24 hours, substantially all of dextran is out of composites leaving behind pores in the osteoimplant composite. An advantage of using dextran in a composite is that dextran exhibits a hemostatic property in extravascular space. Therefore, dextran in a composite can decrease bleeding at or near the site of implantation.

Small molecules including pharmaceutical agents may also be used as porogens in the inventive composites. Examples of polymers that may be used as plasticizers include poly(vinyl pyrollidone), pullulan, poly(glycolide), poly(lactide), and poly(lactide-co-glycolide). Typically low molecular weight polymers are used as porogens. In certain embodiments, a porogen is poly(vinyl pyrrolidone) or a derivative thereof. Plasticizers that are removed faster than the surrounding composite can also be considered porogens.

Components to Deliver

Alternatively or additionally, composites of the present invention may have one or more components to deliver when implanted, including cells, encapsulated cells, biomolecules, small molecules, bioactive agents, etc., to promote bone growth and connective tissue regeneration, and/or to accelerate healing. Examples of materials that can be incorporated include chemotactic factors, angiogenic factors, bone cell inducers and stimulators, including the general class of cytokines such as the TGF-β superfamily of bone growth factors, the family of bone morphogenic proteins, osteoinductors, and/or bone marrow or bone forming precursor cells, isolated using standard techniques. Sources and amounts of such materials that can be included are known to those skilled in the art.

Biologically active materials, comprising biomolecules, small molecules, and bioactive agents may also be included in inventive composites to, for example, stimulate particular metabolic functions, recruit cells, or reduce inflammation. For example, nucleic acid vectors, including plasmids and viral vectors, that will be introduced into the patient's cells and cause the production of growth factors such as bone morphogenetic proteins may be included in a composite. Biologically active agents include, but are not limited to, antiviral agent, antimicrobial agent, antibiotic agent, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, anti-inflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, mesenchymal stem cells, bone digester, antitumor agent, cellular attractant, fibronectin, growth hormone cellular attachment agent, immunosuppressant, nucleic acid, surface active agent, hydroxyapatite, and penetraction enhancer. Additional exemplary substances include chemotactic factors, angiogenic factors, analgesics, antibiotics, anti-inflammatory agents, bone morphogenic proteins, and other growth factors that promote cell-directed degradation or remodeling of the polymer phase of the composite and/or development of new tissue (e.g., bone). RNAi or other technologies may also be used to reduce the production of various factors.

In some embodiments, inventive composites include antibiotics. Antibiotics may be bacteriocidial or bacteriostatic. An anti-microbial agent may be included in composites. For example, anti-viral agents, anti-protazoal agents, anti-parasitic agents, etc. may be include in composites. Other suitable biostatic/biocidal agents include antibiotics, povidone, sugars, and mixtures thereof. Exemplary antibiotics include, but not limit to, Amikacin, Gentamicin, Kanamycin, Neomycin, Netilmicin, Streptomycin, Tobramycin, Paromomycin, Geldanamycin, Herbimycin, Loravabef, etc. (See, The Merck Manual of Medical Information—Home Edition, 1999).

Inventive composites may also be seeded with cells. In some embodiments, a patient's own cells are obtained and used in inventive composites. Certain types of cells (e.g., osteoblasts, fibroblasts, stem cells, cells of the osteoblast lineage, etc.) may be selected for use in the composite. Cells may be harvested from marrow, blood, fat, bone, muscle, connective tissue, skin, or other tissues or organs. In some embodiments, a patient's own cells may be harvested, optionally selected, expanded, and used in the inventive composite. In other embodiments, a patient's cells may be harvested, selected without expansion, and used in the inventive composite. Alternatively, exogenous cells may be employed. Exemplary cells for use with the invention include mesenchymal stem cells and connective tissue cells, including osteoblasts, osteoclasts, fibroblasts, preosteoblasts, and partially differentiated cells of the osteoblast lineage. Cells may be genetically engineered. For example, cells may be engineered to produce a bone morphogenic protein.

In some embodiments, inventive composites may include a composite material comprising a component to deliver. For example, a composite materials can be a biomolecule (e.g., a protein) encapsulated in a polymeric microsphere or nanoparticles. In certain embodiments, BMP-2 encapsulated in PLGA microspheres may be embedded in a bone/polyurethane composite used in accordance with the present invention. Sustained release of BMP-2 can be achieved due to the diffusional barriers presented by both the PLGA and Polyurethane of the inventive composite. Thus, release kinetics of growth factors (e.g., BMP-2) can be tuned by varying size of PLGA microspheres and porosity of polyurethane composite.

To enhance biodegradation in vivo, composites of the present invention can also include different enzymes. Examples of suitable enzymes or similar reagents are proteases or hydrolases with ester-hydrolyzing capabilities. Such enzymes include, but are not limited to, proteinase K, bromelaine, pronase E, cellulase, dextranase, elastase, plasmin streptokinase, trypsin, chymotrypsin, papain, chymopapain, collagenase, subtilisin, chlostridopeptidase A, ficin, carboxypeptidase A, pectinase, pectinesterase, an oxireductase, an oxidase, or the like. The inclusion of an appropriate amount of such a degradation enhancing agent can be used to regulate implant duration.

Components to deliver may not be covalently bonded to a component of the composite. In some embodiments, components may be selectively distributed on or near the surface of inventive composites using the layering techniques described above. While surface of inventive composite will be mixed somewhat as the composite is manipulated in implant site, thickness of the surface layer will ensure that at least a portion of the surface layer of the composite remains at surface of the implant. Alternatively or in addition, biologically active components may be covalently linked to the bone particles before combination with the polymer. As discussed above, for example, silane coupling agents having amine, carboxyl, hydroxyl, or mercapto groups may be attached to the bone particles through the silane and then to reactive groups on a biomolecule, small molecule, or bioactive agent.

Preparation of Composite

In general, inventive composites are prepared by combining particles, polymers and optionally any additional components. To form inventive composites, particles as discussed herein may be combined with a reactive liquid (i.e., a two-component composition) thereby forming a naturally injectable or moldable composite or a composite that can be made injectable or moldable. Alternatively, particles may be combined with polyisocyanate prepolymers or polyols first and then combined with other components.

In some embodiments, particles may be combined first with a hardener that includes polyols, water, catalysts and optionally a solvent, a diluent, a stabilizer, a porogen, a plasticizer, etc., and then combined with a polyisocyanate prepolymer. In some embodiments, a hardener (e.g., a polyol, water and a catalyst) may be mixed with a prepolymer, followed by addition of particles. In some embodiments, in order to enhance storage stability of two-component compositions, the two (liquid) component process may be modified to an alternative three (liquid)-component process wherein a catalyst and water may be dissolved in a solution separating from reactive polyols. For example, polyester polyols may be first mixed with a solution of a catalyst and water, followed by addition of bone particles, and finally addition of NCO-terminated prepolymers.

In some embodiments, additional components or components to be delivered may be combined with a reactive liquid prior to injection. In some embodiments, they may be combined with one of polymer precursors (i.e., prepolymers and polyols) prior to mixing the precursors in forming of a reactive liquid/paste.

Porous composites can be prepared by incorporating a small amount (e.g., <5 wt %) of water which reacts with prepolymers to form carbon dioxide, a biocompativle blowing agent. Resulting reactive liquid/paste may be injectable through a 12-ga syringe needle into molds or targeted site to set in situ. In some embodiments, gel time is great than 3 min, 4 min, 5 min, 6 min, 7 min, or 8 min. In some embodiments, cure time is less than 20 min, 18 min, 16 min, 14 min, 12 min, or 10 min.

In some embodiments, catalysts can be used to assist forming porous composites. In general, the more blowing catalyst used, the high porosity of inventive composites may be achieved. In certain embodiments, surprisingly, surface demineralized bone particles may have a dramatic effect on the porosity. Without being bound to any theory, it is believed that the lower porosities achieved with surface demineralized bone particles in the absence of blowing catalysts result from adsorption of water to the hygroscopic demineralized layer on the surface of bones.

Polymers and particles may be combined by any method known to those skilled in the art. For example, a homogenous mixture of polymers and/or polymer precursors (e.g., prepolymers, polyols, etc.) and particles may be pressed together at ambient or elevated temperatures. At elevated temperatures, a process may also be accomplished without pressure. In some embodiments, polymers or precursors are not held at a temperature of greater than approximately 60° C. for a significant time during mixing to prevent thermal damage to any biological component (e.g., growth factors or cells) of a composite. In some embodiments, temperature is not a concern because particles and polymer precursors used in the present invention have a low reaction exotherm.

Alternatively or in addition, particles may be mixed or folded into a polymer softened by heat or a solvent. Alternatively, a moldable polymer may be formed into a sheet that is then covered with a layer of particles. Particles may then be forced into the polymer sheet using pressure. In another embodiment, particles are individually coated with polymers or polymer precursors, for example, using a tumbler, spray coater, or a fluidized bed, before being mixed with a larger quantity of polymer. This facilitates even coating of the particles and improves integration of the particles and polymer component of the composite.

After combination with particles, polymers may be further modified by further cross-linking or polymerization to form a composite in which the polymer is covalently linked to the particles. In some embodiments, composition hardens in a solvent-free condition. In some embodiments, compositions are a polymer/solvent mixture that hardens when a solvent is removed (e.g., when a solvent is allowed to evaporate or diffuse away). Exemplary solvents include but are not limited to alcohols (e.g., methanol, ethanol, propanol, butanol, hexanol, etc.), water, saline, DMF, DMSO, glycerol, and PEG. In certain embodiments, a solvent is a biological fluid such as blood, plasma, serum, marrow, etc. In certain embodiments, an inventive composite is heated above the melting or glass transition temperature of one or more of its components and becomes set after implantation as it cools. In certain embodiments, an inventive composite is set by exposing a composite to a heat source, or irradiating it with microwaves, IR rays, or UV light. Particles may also be mixed with a polymer that is sufficiently pliable to combine with the particles but that may require further treatment, for example, combination with a solvent or heating, to become a injectable or moldable composition. For example, a composition may be combined and injection molded, injected, extruded, laminated, sheet formed, foamed, or processed using other techniques known to those skilled in the art. In some embodiments, reaction injection molding methods, in which polymer precursors (e.g., polyisocyanate prepolymer, a polyol) are separately charged into a mold under precisely defined conditions, may be employed. For example, bone particles may be added to a precursor, or it may be separately charged into a mold and precursor materials added afterwards. Careful control of relative amounts of various components and reaction conditions may be desired to limit the amount of unreacted material in a composite. Post-cure processes known to those skilled in the art may also be employed. A partially polymerized polyurethane precursor may be more completely polymerized or cross-linked after combination with hydroxylated or aminated materials or included materials (e.g., a particulate, any components to deliver, etc.).

In some embodiments, an inventive composite is produced with a injectable composition and then set in situ. For example, cross-link density of a low molecular weight polymer may be increased by exposing it to electromagnetic radiation (e.g., UV light) or an alternative energy source. Alternatively or additionally, a photoactive cross-linking agent, chemical cross-linking agent, additional monomer, or combinations thereof may be mixed into inventive composites. Exposure to UV light after a composition is injected into an implant site will increase one or both of molecular weight and cross-link density, stiffening polymers (i.e., polyurethanes) and thereby a composite. Polymer components of inventive composites used in the present invention may be softened by a solvent, e.g., ethanol. If a biocompatible solvent is used, polyurethanes may be hardened in situ. In some embodiments, as a composite sets, solvent leaving the composite is released into surrounding tissue without causing undesirable side effects such as irritation or an inflammatory response. In some embodiments, compositions utilized in the present invention becomes moldable at an elevated temperature into a pre-determined shape. Composites may become set when composites are implanted and allowed to cool to body temperature (approximately 37° C.).

The invention also provides methods of preparing inventive composites by combining cells encapsulated in gel beads and polyurethane precursors and resulting in naturally flowable compositions. Alternatively or additionally, the invention provides methods to make a porous composite include adding a solvent or pharmaceutically acceptable excipient to render a flowable or moldable composition. Such a composition may then be injected or placed into the site of implantation. As solvent or excipient diffuses out of the composite, it may become set in place.

In some embodiments, cells may be mixed with a polymer precursor according to standard composite processing techniques. For example, encapsulated cells may simply be suspended in a precursor. A polymer precursor may be mechanically stirred to distribute the cells or bubbled with a gas, preferably one that is oxygen- and moisture-free. Once components of a composition are mixed, it may be desirable to store it in a container that imparts a static pressure to prevent separation of the particles and the polymer precursor, which may have different densities. In some embodiments, distribution and cell/polymer ratio may be optimized to produce at least one continuous path through a composite along cells.

Inventive composites utilized in the present invention may include practically any ratio of polyurethane and cells (e.g., cells encapsulated in gel beads), for example, between about 5 wt % and about 95 wt % cells encapsulated in gel beads. In some embodiments, composites may include about 40 wt % to about 45 wt % cells encapsulated in gel beads, about 45 wt % to about 50 wt % cells encapsulated in gel beads or about 50 wt % to about 55 wt % cells encapsulated in gel beads. In some embodiments, composites may include about 55 wt % to about 70 wt % cells encapsulated in gel beads. In some embodiments, composites may include about 70 wt % to about 90 wt % cells encapsulated in gel beads. In some embodiments, composites may include at least approximately 40 wt %, 45 wt %, 50 wt %, or 55 wt % of cells encapsulated in gel beads. In certain embodiments, such weight percentages refer to weight of cells encapsulated in gel beads and other particulates desired to be included in the composite.

In some embodiments, composites may include at least approximately 30 vol %, 35 vol %, 40 vol %, or 50 vol % of cells encapsulated in gel beads. In some embodiments, a volume percentage of cells encapsulated in gel beads in composite in accordance with the present invention may be about 30 vol %, 35 vol %, 40 vol %, 50 vol %, 60 vol %, 70 vol % or between any volume percentages of above. In some embodiments, injectable composites in accordance with the present invention may have a volume percentage (fraction) of at least approximately 36 vol % of cells encapsulated in gel beads and/or other particulate materials. In some embodiments, volume percentages (fractions) of cells encapsulated in gel beads and/or other particulate materials in porous composites in the present invention may be less than 64 vol %. In certain embodiments, for a certain volume percentage, corresponding weight percentage of cells encapsulated in gel beads and/or other particulate materials varies depending on density of cells encapsulated in gel beads.

Desired proportion may depend on factors such as injection sites, shape and size of the particles, how evenly polymer is distributed among particles, desired flowability of composites, desired handling of composites, desired moldability of composites, and mechanical and degradation properties of composites. The proportions of polymers and particles can influence various characteristics of the composite, for example, its mechanical properties, including fatigue strength, the degradation rate, and the rate of biological incorporation. In addition, the cellular response to the composite will vary with the proportion of polymer and particles. In some embodiments, the desired proportion of particles may be determined not only by the desired biological properties of the injected material but by the desired mechanical properties of the injected material. That is, an increased proportion of particles will increase the viscosity of the composite, making it more difficult to inject or mold. A larger proportion of particles having a wide size distribution may give similar properties to a mixture having a smaller proportion of more evenly sized particles.

Inventive composites of the present invention can exhibit high degrees of porosity over a wide range of effective pore sizes. Thus, composites may have, at once, macroporosity, mesoporosity and microporosity. Macroporosity is characterized by pore diameters greater than about 100 microns. Mesoporosity is characterized by pore diameters between about 100 microns about 10 microns; and microporosity occurs when pores have diameters below about 10 microns. In some embodiments, the composite has a porosity of at least about 30%. For example, in certain embodiments, the composite has a porosity of more than about 50%, more than about 60%, more than about 70%, more than bout 80%, or more than about 90%. In some embodiments, inventive composites have a porosity in a range of 30%-40%, 40%-45%, or 45%-50%. Advantages of a porous composite over non-porous composite include, but are not limited to, more extensive cellular and tissue in-growth into the composite, more continuous supply of nutrients, more thorough infiltration of therapeutics, and enhanced revascularization, allowing bone growth and repair to take place more efficiently. Furthermore, in certain embodiments, the porosity of the composite may be used to load the composite with biologically active agents such as drugs, small molecules, cells, peptides, polynucleotides, growth factors, osteogenic factors, etc, for delivery at the implant site. Porosity may also render certain composites of the present invention compressible.

In some embodiments, pores of inventive composite may be over 100 microns wide for the invasion of cells and bony in-growth (Klaitwatter et al., J. Biomed. Mater. Res. Symp. 2:161, 1971; which is incorporated herein by reference). In certain embodiments, the pore size may be in a ranges of approximately 50 microns to approximately 750 microns, for example, of approximately 100 microns to approximately 500 microns.

In some embodiments, compressive strength of dry inventive composites may be in an approximate range of 4-10 MPa, while compressive modulus may be in an approximate range of 150-450 MPa. Compressive strength of the wet composites may be in an approximate range of 4-13 MPa, while compressive modulus may be in an approximate 50-350 MPa.

After implantation, inventive composites are allowed to remain at the site providing the strength desired while at the same time promoting healing, regeneration, and/or repair of tissue. Polyurethane of composites may be degraded or be resorbed as new tissue is formed at the implantation site. Polymer may be resorbed over approximately 1 month to approximately 1 years. Composites may start to be remodeled in as little as a week as the composite is infiltrated with cells or new tissue in-growth. A remodeling process may continue for weeks, months, or years. For example, polyurethanes used in accordance with the present invention may be resorbed within about 4-8 weeks, 2-6 months, or 6-12 months. A degradation rate is defined as the mass loss as a function of time, and it can be measured by immersing the sample in phosphate buffered saline or medium and measuring the sample mass as a function of time.

One skilled in the art will recognize that standard experimental techniques may be used to test these properties for a range of compositions to optimize a composite for a desired application. For example, standard mechanical testing instruments may be used to test the compressive strength and stiffness of composites. Cells may be cultured on composites for an appropriate period of time, and metabolic products and amount of proliferation (e.g., the number of cells in comparison to the number of cells seeded) may be analyzed. Weight change of composites may be measured after incubation in saline or other fluids. Repeated analysis will demonstrate whether degradation of a composite is linear or not, and mechanical testing of incubated materials will show changes in mechanical properties as a composite degrades. Such testing may also be used to compare enzymatic and non-enzymatic degradation of a composite and to determine levels of enzymatic degradation. A composite that is degraded is transformed into living bone upon implantation.

Use and Application of Composite

As discussed above, polymers or polymer precursors, and other components may be supplied separately, e.g., in a kit, and mixed immediately prior to implantation, injection or molding. A surgeon or other health care professional may also combine components in a kit with autologous tissue derived during surgery or biopsy. For example, a surgeon may want to include autogenous tissue or cells, e.g., bone marrow or bone shavings generated while preparing a implant site, into a composite (see more details in co-owned U.S. Pat. No. 7,291,345 and U.S. Ser. No. 11/625,119 published under No. 2007-0191963; both of which are incorporated herein by reference).

Composites of the present invention may be used in a wide variety of clinical applications. A method of preparing and using polyurethanes for orthopedic applications utilized in the present invention may include the steps of providing a curable cell/polyurethane composition, mixing parts of a composition, and curing a composition in a tissue site wherein a composition is sufficiently flowable to permit injection by minimally invasive techniques. In some embodiments, a flowable composition to inject may be pressed by hand or machine. In some embodiments, a moldable composition may be pre-molded and implanted into a target site. Injectable or moldable compositions utilized in the present invention may be processed (e.g., mixed, pressed, molded, etc.) by hand or machine.

Inventive composites and/or compositions may be used as injectable materials with or without exhibiting high mechanical strength (i.e., load-bearing or non-load bearing, respectively). In some embodiments, inventive composites and/or compositions may be used as moldable materials. For example, compositions (e.g., prepolymer, monomers, reactive liquids/pastes, polymers, bone particles, additional components, etc.) in the present invention can be pre-molded into pre-determined shapes. Upon implantation, the pre-molded composite may further cure in situ and provide mechanical strength (i.e., load-bearing). A few examples of potential applications are discussed in more detail below.

In some embodiments, compositions and/or composites of the present invention may be used as a bone void filler. Bone fractures and defects, which result from trauma, injury, infection, malignancy or developmental malformation can be difficult to heal in certain circumstances. If a defect or gap is larger than a certain critical size, natural bone is unable to bridge or fill the defect or gap. These are several deficiencies that may be associated with the presence of a void in a bone. Bone void may compromise mechanical integrity of bone, making bone potentially susceptible to fracture until void becomes ingrown with native bone. Accordingly, it is of interest to fill such voids with a substance which helps voids to eventually fill with naturally grown bone. Open fractures and defects in practically any bone may be filled with composites according to various embodiments without the need for periosteal flap or other material for retaining a composite in fracture or defect. Even where a composite is not required to bear weight, physiological forces will tend to encourage remodeling of a composite to a shape reminiscent of original tissues.

Many orthopedic, periodontal, neurosurgical, oral and maxillofacial surgical procedures require drilling or cutting into bone in order to harvest autologous implants used in procedures or to create openings for the insertion of implants. In either case voids are created in bones. In addition to all the deficiencies associated with bone void mentioned above, surgically created bone voids may provide an opportunity for incubation and proliferation of any infective agents that are introduced during a surgical procedure. Another common side effect of any surgery is ecchymosis in surrounding tissues which results from bleeding of the traumatized tissues. Finally, surgical trauma to bone and surrounding tissues is known to be a significant source of post-operative pain and inflammation. Surgical bone voids are sometimes filled by the surgeon with autologous bone chips that are generated during trimming of bony ends of a graft to accommodate graft placement, thus accelerating healing. However, the volume of these chips is typically not sufficient to completely fill the void. Composites and/or compositions of the present invention, for example composites comprising anti-infective and/or anti-inflammatory agents, may be used to fill surgically created bone voids.

Inventive composites may be administered to a subject in need thereof using any technique known in the art.

The following examples are presented to be exemplary of certain embodiments of the present invention. They are not to be construed as being limiting thereof.

### EXAMPLES

### Example 1

This example relates to gel microstructure regulation of proliferation and differentiation of MC3T3-E1 cells encapsulated in alginate beads.

Materials and Methods: An O/W emulsion technique was used for encapsulation of cells in alginate beads. Alginic acid (viscosity 20,000˜40,000 cps, Aldrich) was dissolved in Di-water and in α-MEM at the concentrations with 1 and 2% (w/v). MC3T3-E1 embryonic mouse osteoblast precursor cells were suspended in the alginate solutions. Cell alginate suspensions were then added dropwise into two kinds of Ca-catalysts: (a) CaCl₂ in Di-water and (b) CaCl₂ in α-MEM. The concentrations of CaCl₂ solutions were 100 and 200 mM. The beads, thus formed, were cured in the Ca-catalyst medium for 2 hr. MC3T3-E1 encapsulated with 1×10⁶ cells/ml in alginate beads. The viability of encapsulated MC3T3-E1 cells was quantified using the live/dead viability assay. Cell differentiation in the alginate beads was characterized using alkaline phosphatase (ALP) and osteocalcin.

Results: The present inventors have discovered that the cell viability of Ca-alginate beads with α-MEM (>99%) was significantly higher than that of Ca-alginate beads formed with DI-water (<80) after cell encapsulation. It is conjectured that using α-MEM as the solvents for polymer matrix and catalyst solutions to prepare the alginate beads supplied nutrients and oxygen during cell encapsulation. ALP activity and osteocalcin expression of the alginate beads with small mesh pore size (˜100 nm; 2 wt. % alginic solution+200 mM CaCl₂ solution) were significantly higher than that of the alginate beads with larger mesh pore size (˜260 nm; 1 wt. % alginic solution+100 mM CaCl₂ solution). Thus differentiation was dominant in the beads with small mesh pore size during cell culture because of insufficient space for cell proliferation. Total protein in the alginate beads with high mesh pore size increased with time for up to 5 days. However, total protein of the alginate beads with small mesh pore size did not increase significantly until after 15 days. Thus proliferation of the encapsulated cells was dominant in alginate beads with large mesh pore size.

The overall results show that cellular outcomes in alginate beads can be regulated by internal pore structure. This approach may be utilized to design cell transplantation vehicles that both localize and maintain the viability of the transplanted cells, as well as regulate cell proliferation and differentiation.

### Example 2

This Example also relates to the changes in proliferation and differentiation of MC3T3-E1 cells in alginate beads with differing mesh microstructures.

Materials and Methods

Alginic acid sodium salt (viscosity 20,000˜40,000 cP, molecular weight 120-190 kDa, Aldrich, St. Louis, Mo.) was dissolved in DI-water and in α-MEM at concentrations of 1 and 2% (w/v). Briefly, MC3T3-E1 cells (1×10⁶ cells/ml) were suspended in the alginic acid sodium salt solutions (1 and 2% (w/v)) and mixed for 2 h. Cell viability was measured by live/dead staining before and after incubation in alginic acid solution and found to be unchanged. Suspensions of cells in alginate were then added drop-wise into two different crosslinker solutions: (a) CaCl₂ in DI-water or (b) CaCl₂ in α-minimum essential medium (MEM) at room temperature. The concentration of CaCl₂ solutions was either 100 or 200 mM. The beads formed in the microencapsulation device were subsequently cured in the CaCl₂ solution for 1 h. Each formulation of alginate beads was designated as A-B, where A represents the concentration of the alginate solution (1 or 2 wt %) and B denotes the concentration of CaCl₂ solution (100 or 200 mM). For example, composition 1-100 corresponds to alginate beads synthesized with 1 wt % alginate solution and 100 mM CaCl₂ solution.

Four alginate bead compositions were investigated as summarized in Table 1.

TABLE 1
Composition and initial properties of alginate beads.

| | Alginate | CaCl₂ | ν₂ | M_{c}, g/mol | | Mesh size (ξ), nm | |
|---|---|---|---|---|---|---|---|
| Bead | wt % | mM | vol % | Swelling | Rheology | Swelling | Rheology |
| 1-100 | 1.0 | 100 | 0.239 ± 0.018 | 2674 ± 617 | 6518 ± 1287 | 19.3 ± 2.7 | 30.0 ± 2.7 |
| 1-200 | 1.0 | 200 | 0.264 ± 0.003 | 1921 ± 59 | 4510 ± 670 | 15.8 ± 0.3 | 24.2 ± 1.7 |
| 2-100 | 2.0 | 100 | 0.351 ± 0.011 | 781 ± 85 | 3942 ± 346 | 9.2 ± 0.6 | 14.2 ± 0.8 |
| 2-200 | 2.0 | 200 | 0.513 ± 0.021 | 211 ± 30 | 2692 ± 6 | 4.2 ± 0.4 | 10.6 ± 0.1 |

The alginate solution was added drop-wise into the CaCl₂ solution in DI-water (100 or 200 mM). Images of 30 beads obtained by optical microscopy (OM) were analyzed using an Olympus DP71 camera attached to a fluorescent microscope (Olympus CKX41, U-RFLT50, Center Valley, Pa.) to measure bead size and analyze cell morphology. The beads were incubated in DI-water at 37° C. and bead size measured as a function of time for up to 10 days. The swelling ratio and mesh size of each alginate bead composition were calculated from swelling experiments [39-43]. Five beads were incubated in DI-water at 37° C., dried under vacuum, and weighed. The swelling ratio q_{F} and volume fraction polymer ν₂ were calculated from Eqs. (1) and (2) [44]:

$$\begin{matrix}{q}_{F} = \frac{mass of gel after preparation ( wet )}{mass of gel after drying ( dry )} & ( 1 ) \\ {v}_{2} = 1 + ( {q}_{F} - 1 ) {ρ}_{P} / {ρ}_{W} & ( 2 )\end{matrix}$$

where ρ_{P} is the density of the alginate polymer (1.601 g/cm³) and ρ_{W} is the density of water (1.0 g/cm³).

The crosslink density n was calculated from the Flory-Rehner equation (Eq. (3)) [39, 40]:

n=−[ln(1−ν₂)+ν₂+χ₁ν₂]/[V₁(ν₂^{1/3}−0.5ν₂) (3)

where χ₁ is the Flory-Huggins interaction parameter (˜0.5), V₁ is the molar volume of the solvent (water, 18 cm³), and n represents the number of active network chain segments per unit volume (mol cm⁻³) [43, 45]. The molecular weight between crosslinks is then given by Eq. (4):

M_{c}=ρ_{P}/n (4)

Due to the high density of the alginate network, permeability experiments for measuring the pore size in the gel were not feasible [41]. The corresponding mesh size, ξ, can be subsequently approximated using Eq. (5) [39-43]:

ξ=ν₂^{-1/3}l*(2M_{c}/Mᵣ)^{1/2}Cₙ^{1/2} (5)

where Mᵣ is the molecular weight (390.1 g/mol) of the repeat unit, 1 is the carbon-carbon bond length of monomer unit (assumed to be 5.15 Å), and Cₙ is the characteristic ratio for alginate calculated as Cₙ=0.021*Mₙ+17.95=21.1 [43, 45]. The mesh size effectively represents the maximum diameter of a molecule that can diffuse through the ideal network.

Cells encapsulated in alginate beads were cultured in either standard culture medium or osteogenic culture medium, which consisted of α-MEM, 2.5% (v/v) FBS, 1% (v/v) penicillin (100 U/ml)/streptomycin (100 μg/ml), 50 μg/ml L-ascorbic acid, and 10 mM β-glycerophosphate, in the incubator with 5% CO₂ at 37° C. Cell culture was performed under dynamic conditions using an orbital shaker operated at 100 rpm at 37° C. Sample analysis or preparation for qualitative assessment performed on days 1, 2, 5, 10, and 15. The medium was replaced every 2 days, and the cells were recovered from the beads as described below.

The storage and loss moduli of alginate gels were measured under shear conditions at 25° C. using a TA Instruments AR-G2 Rheometer (TA Instruments, New Castle, Del.) fitted with parallel plates (diameter of 25 mm, gap of 1 mm) [46]. Disk-shaped alginate gels were prepared by adding 1 ml alginic acid sodium salt solution (1 or 2% (w/v) in DI-water) to a mixing cup and reacted with 10 ml of CaCl₂ crosslinker solution in DI-water (100 or 200 μM). The alginate gels were fully reacted for 12 h at room temperature. Shear storage and loss moduli were measured by oscillatory shear experiments performed with strain amplitude of 1.5% and oscillation frequency of 10 rad/s, which is within the linear viscoelasticity region as verified by frequency and strain sweeps following each measurement. The molecular weight between crosslinks was calculated from the measured value of the shear modulus (G) using the following equation [24, 47]:

$${M}_{c , R} = \frac{{c}_{P} RT}{G} \qquad ( 6 )$$

where c_{P} is the concentration of alginate in solution (1 or 2 wt %), R is the gas constant (8.314 m³ Pa mol⁻¹ K⁻¹), and T is the temperature.

4 mM cell-permeable calcein acetoxymethyl (Calcein AM) and 2 mM ethidium homodimer-1 (EthD-1) from the Live/Dead Viability/Cytotoxicity Kit (Invitrogen) for mammalian cells was added to the samples. Calcein AM produces a bright green fluorescence in live cells. Ethidium homodimer-1 is retained within damaged or dead cells, imparting a bright red fluorescence. Fluorescence images were observed by an Olympus DP71 camera attached to a fluorescent microscope (Olympus CKX41, U-RFLT50, Center Valley, Pa.). Cell viability (the percentage of viable cells) was measured by counting the numbers of live and dead cells from the fluorescent images at time points of 0, 1, 2, 5, and 10 days.

Five alginate beads were harvested from 24-well plates at days 1, 2, 5, 10, and 15 and immersed in phosphate-buffered saline (PBS) for 5 min followed by washing with PBS three times. The washed beads were crushed and lysed with 150 μl of 0.05% Triton X-100. The plates were homogenized by three freeze-thaw cycles. After removing the lysate, the alginate fragments were viewed in the microscope and no evidence of cells was apparent. The lysates (20 μl) were added to 96-well plates with 100 μl substrate buffer (2 mg/ml disodium p-nitrophenylphosphate hexahydrate and 0.75 M 2-amino-2-methyl-1-propanol). The mixtures were then incubated for 30 min at 37° C. and the resulting optical absorbance measured at 410 nm by using a mQuant spectrophotometer (Bio-Tek Instruments Inc.). ALP activity was determined from a standard curve generated by employing the reaction of a p-nitrophenyl solution. The ALP activity was normalized using the measured total protein to account for differences in the number of cells on different alginate beads at individual time points. Total cellular protein was determined with a BCA protein assay kit (Pierce). The lysates (10 μl) were mixed with 200 μl BCA working reagent containing cupric sulfate and bicinchoninic acid in 96-well plates, and then incubated for 30 min at 37° C. The resulting optical absorbance was measured at 562 nm with the μQuant spectrophotometer. Total protein amounts were calculated with a standard curve, which was generated with bovine serum albumin.

Osteocalcin (OCN) secretion from encapsulated MC3T3-E1 cells in alginate beads was measured as a later marker of osteogenic differentiation. Secreted OCN was measured from the standard and osteogenic culture media at 16 days by an enzyme immuno assay (mouse osteocalcin EIA kit BT 470, Biomedical Technologies, Inc.) using the instructions in the kit. The absorbance was measured at 450 nm with the μQuant spectrophotometer. Mouse OCN was used to generate a standard curve, which was prepared from 7 OCN standards. The concentration of OCN was determined by interpolation and normalized by total protein.

The rates of proliferation and differentiation were calculated from the total protein (TP, mg/ml) and alkaline phosphatase activity normalized by total protein (ALP/TP, U/(mg*ml)) versus time data:

$${r}_{P} = \frac{d}{d t} ( TP ) \\ {r}_{D} = \frac{d}{d t} ( ALP / TP ) \qquad ( 7 )$$

Both TP and ALP/TP data were assumed to be linear functions of time, and thus the values of r_{P} and r_{D} were calculated from the slopes of the TP and ALP/TP versus time plots, respectively. The ratio of the dimensionless proliferation rate to the dimensionless differentiation rate was calculated as follows:

$${r}_{P / D} = \frac{{r}_{P}^{′}}{{r}_{D}^{′}} = \frac{{r}_{P} / {TP}_{1}}{{r}_{D} / {( ALP / TP )}_{1}} \qquad ( 8 )$$

where TP₁ and (ALP/TP)ᵢ denote the TP and ALP/TP values measured on day 1.

All data are presented as mean standard deviation (±S.D.). One way ANOVA with bonferroni correction (p<0.05) was used for evaluation of statistical significance for all data.

Results

The viability of cells encapsulated in beads prepared with α-MEM solvents during the synthesis process exceeded 98%, while in the beads synthesized with DI water solutions the viability was <65%. When synthesized in the presence of DI water, cells are exposed to a nutrient-limited environment for at least 4 h, which is conjectured to result in increased cell death compared to synthesis in the presence of α-MEM.

Gels with a larger mesh size and lower shear modulus to facilitated proliferation, while gels with a smaller mesh size spatially constrained the cells and promoted differentiation. The initial sizes of the 1-100, 1-200, 2-100 and 2-200 alginate beads measured by image analysis were 2.072±0.026, 2.085±0.028, 2.263±0.029 and 2.201±0.028 mm, respectively. As shown in FIG. 1A, the diameter of all four compositions of alginate beads increased from day 1 to day 10. FIG. 1B displays the mass swelling ratio as a function of the immersion time in DI water for all four alginate compositions. Consistent with the increase in bead size with time, the swelling ratio increased with time for all four compositions. The swelling ratio is related to the crosslink density by the Flory-Rehner equation, which predicts that swelling increases with the molecular weight between crosslinks. While mammals lack the enzyme alginase that cleaves alginate chains, ionically cross-linked alginate gels dissolve by release of the divalent Ca²⁺ ions into the surrounding media resulting from cation exchange with monovalent sodium ions [48]. Thus, when the beads are immersed in DI water, the Ca²⁺ crosslinks break down, resulting in an increase in the molecular weight between crosslinks. The initial mesh sizes (ξ) of the 1-100, 1-200, 2-100 and 2-200 alginate beads were 19.8±2.8, 16.3±0.3, 9.4±0.6 and 4.3±0.4 nm, respectively. As expected, the calculated gel mesh size (FIG. 1C) decreased with increasing concentration of both alginate and calcium solutions, and increased with time when incubated in DI water for 10 days (FIG. 1C). For example, the mesh size of 1-100 beads increased from 19.8±2.8 nm to 240.2±18.9 nm after 10 days of incubation in DI-water. In contrast, the mesh size of 2-200 beads showed a substantially smaller increase in mesh size (4.3 to 33.8 nm) compared to the 1-100 beads.

However, because the Flory-Rehner equation makes several assumptions, the mesh size calculated from Eqs (5) and (6) is an approximation of the actual pore size in an alginate gel [41]. While the mesh size decreased with M_{c}, the shear modulus G of the gels increased with decreasing M_{c}. Also, the limited applicability of the Flory-Rehner theory to alginate gels may contribute to the discrepancy in initial mesh sizes measured by the swelling and rheology experiments.

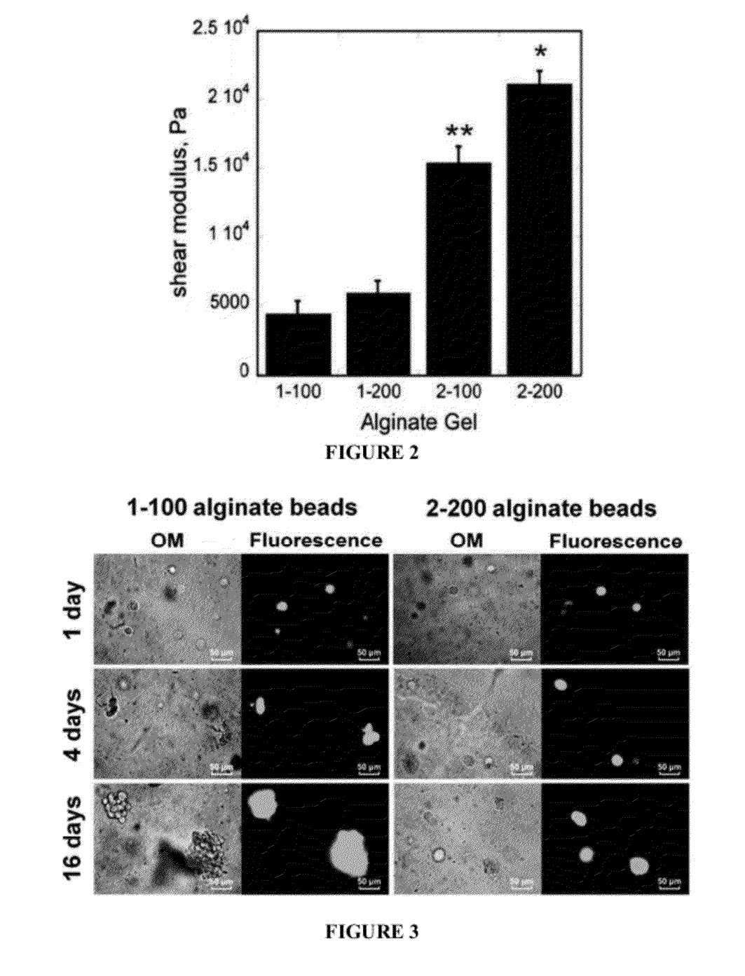

The alginate composition also controlled the initial mechanical properties of the gels, as evidenced by the increase in shear modulus with increasing calcium and alginate concentration (FIG. 2). For example, formulation 1-100 had the lowest elastic modulus (3.91 kPa), while formulation 2-200 had the highest elastic modulus (18.4 kPa). As shown by Eq (6), the shear modulus scales with M_{c}⁻¹ and ξ^{-1/2}. The initial values of ξ calculated from the initial shear modulus data are ˜50% greater than those calculated from the swelling data (Table 1). Thus the microstructure and mechanical properties of the gels are inter-related and can be controlled by changing the concentrations of the alginate and CaCl₂ solutions.

To investigate the effects of gel microstructure on cell fate, we performed optical microscopy (OM) with fluorescent staining Representative OM and live/dead (L/D) staining images of 1-100 alginate beads show that low-density alginate beads characterized by a larger mesh size supported proliferation of the encapsulated MC3T3-E1 cells (FIG. 3, 1-100 alginate beads). Mitosis began after day 1, and small clusters of cells were observed after day 4 (FIG. 3, 1-100 alginate beads). At 16 days, large clusters of MC3T3-E1 cells formed as a result of proliferation (FIG. 3, 1-100 alginate beads). There was no evidence of apoptotic features such as cell shrinkage or dead cells (red fluorescence) in the clusters. In contrast, the high-density 2-200 beads did not reveal evidence of clustering at day 1, 4, or 10 (FIG. 3, 2-200 alginate beads).

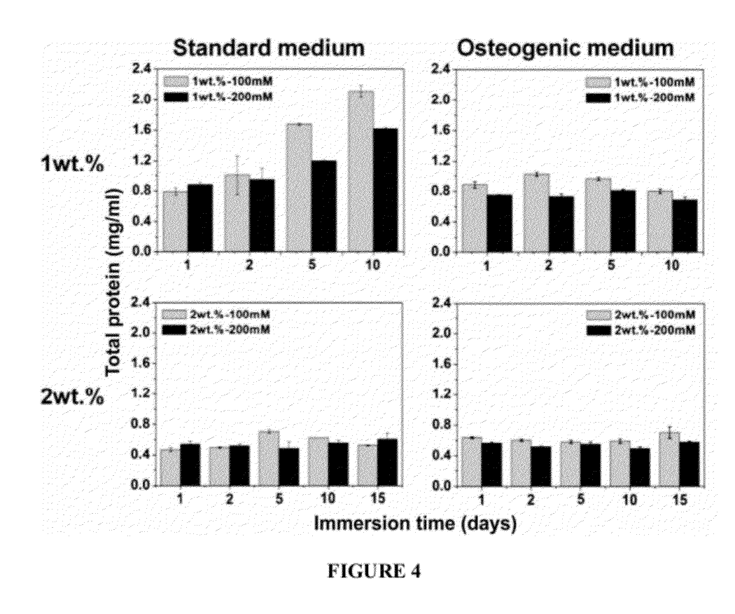

FIG. 4 presents total protein measured in 5 alginate beads cultured under dynamic conditions as a function of immersion time in the standard and osteogenic culture media. Total protein in the low-density alginate beads (1-100 and 1-200) increased significantly over the culture period. Furthermore, at each time point there was significantly more total protein in the less crosslinked, compliant 1-100 alginate beads than in the 2-200 alginate beads (P<0.05). In contrast, total protein measured for the high-density alginate beads (2-100 and 2-200) with small mesh size did not increase significantly up to 15 days (FIG. 4, 2 wt % row). Thus proliferation of the encapsulated cells was greatest in the alginate beads with the lowest density, highest shear modulus, and largest large mesh size.

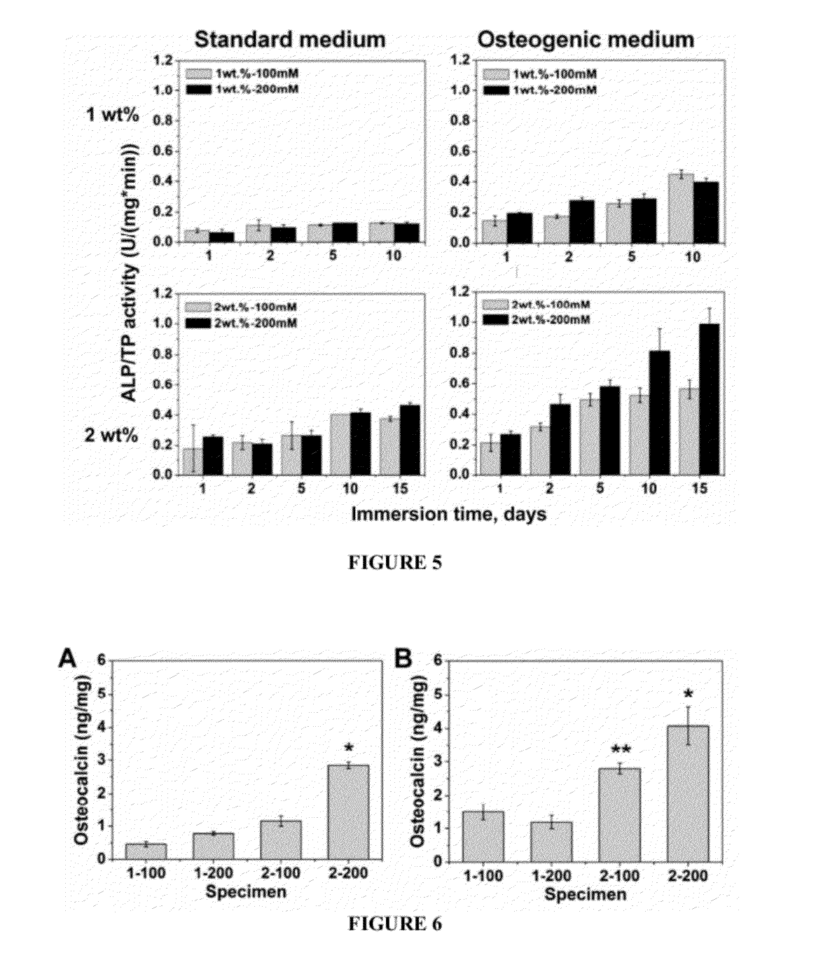

Alkaline phosphatase (ALP) activity and osteocalcin (OCN) assays were used to evaluate osteogenic differentiation of the encapsulated MC3T3-E1 cells in the four types of alginate beads (FIGS. 5-6). ALP and OCN are early- and late-stage markers of osteoblast differentiation, respectively [32]. By day 15, the 1-100 and 1-200 beads had degraded and fractured under dynamic cell culture conditions using the standard culture medium, resulting in release of cells which subsequently attached to the well plate. Thus it was not possible to measure total protein and ALP activity after 15 days for the 1-100 and 1-200 beads. As shown in FIG. 5, in standard culture medium ALP activity (normalized by total protein, TP) was relatively constant with time at ˜0.1 U/mg/min in 1-100 and 1-200 beads, while in 2-100 and 2-200 beads it increased with time up to >0.4 U/mg/min. Similarly, OCN expression in the 2-200 beads with the smallest initial mesh size (4.3 nm) was significantly higher than that in all other beads as shown in FIG. 6A (P<0.05). In osteogenic medium, ALP/TP increased with time for all groups, and activity was higher in the 2-100 and 2-200 beads. Similarly, OCN expression was significantly higher for each treatment group when cultured in osteogenic medium compared to standard medium, and was significantly higher in 2-100 and 2-200 beads compared to the 1 wt % alginate beads (FIG. 6B). Taken together, the data in FIGS. 5 and 6 suggest that differentiation was dominant in rigid low-density beads with small mesh size, while proliferation was dominant in compliant low-density beads with larger mesh size when cells were cultured in α-MEM.

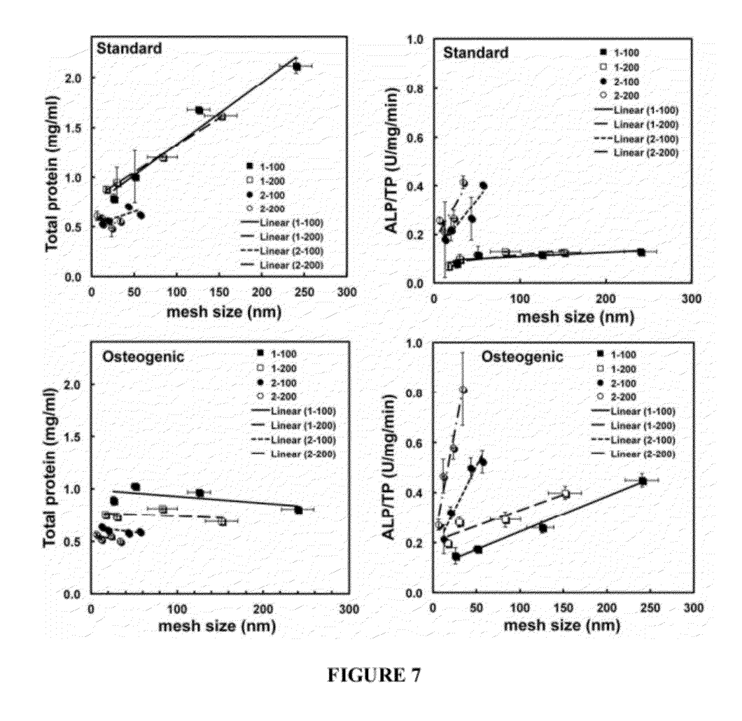

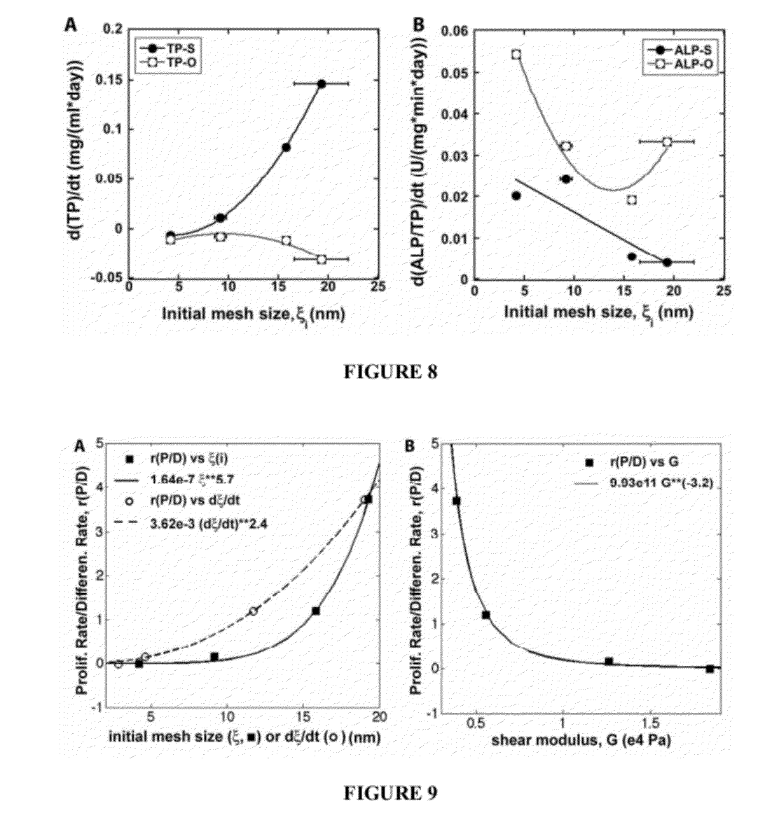

Total protein (TP, mg/ml) and ALP/TP (U/mg/min) are plotted versus the initial mesh size in FIG. 7. In standard medium, TP increases with mesh size for the 1-100 and 1-200 gels, while it is relatively constant for the 2-100 and 2-200 gels. In contrast, ALP/TP increases with initial mesh size for the 2-100 and 2-200 gels, while it is relatively constant for the 1-100 and 1-200 gels. In osteogenic medium, TP is relatively constant with mesh size and ALP/TP increases with mesh size. However, the increase in mesh size ξ with time due to dissolution of the gel confounds the interpretation of the proliferation and differentiation data shown in FIG. 7. To more clearly show the effects of initial mesh size on cell fate, the rates of proliferation and differentiation calculated from Eq (7) are plotted versus initial mesh size in FIG. 8. The proliferation rate r_{P} increases dramatically with initial mesh size, while the differentiation rate r_{D} decreases strongly with increasing initial mesh size in standard medium. These observations support the notion that the relatively large (e.g., >16 nm) initial mesh size of the 1-100 and 1-200 gels supports cell proliferation, while the smaller (e.g., <10 nm) initial mesh size of the 2-100 and 2-200 gels supports cell differentiation. In osteogenic medium, the differentiation rate decreases with initial mesh size, which suggests that the mesh size regulates differentiation even in the presence of osteogenic medium.

### Example 3

This example relates to local cell delivery from injectable biodegradable polymeric scaffolds.

Materials and Methods: Similarly to Examples 1 and 2, an O/W emulsion technique was used for encapsulation of cells in alginate beads. MC3T3-E1 embryonic mouse osteoblast precursor cells were encapsulated with 1×10⁶ cells/ml in alginate beads. Alginate beads were prepared using Ca-catalysts as CaCl₂ in α-MEM. The loading of cell-encapsulated beads in the reactive PUR scaffold was 50 wt %. Cell survivability in alginate beads was determined using live/dead staining Cell differentiation in the alginate beads, beads alone, and beads incorporated in PUR, was characterized using alkaline phosphatase (ALP) and osteocalcin.

Results: Biomimetic cell-alginate capsules are successfully synthesized by O/W emulsion technique. The synthesized Ca-alginate/PUR composite exhibited three different pore structures: macropores (0.5˜2 mm) from degradation of alginate beads, intermediate pores (˜200 μm), and micropores (several μm) in the PUR matrix. We have shown that encapsulating MC3T3-E1 cells in α-MEM alginate solution protects the cells from the polymerization and improves cell viability. The injectable Ca-alginate/PUR composite scaffold also supports differentiation of MC3T3-E1 preosteoblast cells. Without being bound by theory or mechanism, α-MEM in alginate beads supplies nutrients and oxygen for the encapsulated cells during PUR polymerization, and connectivity between Ca-alginate beads facilitates transport of nutrients after polymerization.

The overall results indicate that incorporation of α-MEM within alginate beads can improve cell survivability. Injectable cell-alginate/PUR scaffolds of the present invention are useful for healing of massive tissue defects. Furthermore, this approach can be applied for various cell therapies.

### Example 4

This Example relates to biodegradable composites comprising a polyurethane component and alginate encapsulated MC3T3 cells, as well as the biological and mechanical characteristics of such composites.

Materials and Methods

Calcium alginate hydrogel was used to encapsulate and protect cells. Alginate hydrogel was prepared by the reaction between sodium alginate acid (Aldrich Chemistry, viscosity=20-40 cps) solution of 1-2 w/v % and gelling agent solution. A 100 mM-200 mM calcium chloride (Acros Organics) solution was used as the gelling agent solution. The formation process of spherical gel beads is to drip droplets of sodium alginate solution through a nozzle (diameter=0.35 μm) into calcium chloride solution using a syringe pump (rate=10 mL/h). The synthesis process was carried out at room temperature. An electronic bead maker (Nisco, VAR V1) was used to control bead size by charging the nozzle with a high DC voltage [10] so that there was potential difference between the nozzle and gelling agent solution. Alginate bead size was determined by a light microscope (Olympus BX60).

MC3T3 from Mus musculus was used as the encapsulated cell line in this study. Cells were cultured in a complete medium of αMEM (GIBCO) with 10% fetal bovine serum (FBS) and 1% penicillin/streptomycin (PS). At 80-90% confluence, MC3T3 cells were detached by trypsin (0.25%). After suspending in alginate solution for 1 h, MC3T3 were encapsulated in hydrogel at a density of 5×10⁵ cells/mL of alginate solution. In cell encapsulation process, the complete medium described above was used as the solvent of both sodium alginate and calcium chloride solutions.

To partially oxidize sodium alginate, an aqueous solution of sodium periodate (0.25M, 2.0 mM) was mixed with 1 w/v % solution of sodium alginate at room temperature. After reacting in dark bottle for 24 h, two drops of ethylene glycol were added to stop the oxidation. The resultant solution was precipitated with ethanol (2:1, v/v, ethanol/water) with the existence of sodium chloride (6.25 g/L). Collected precipitates were redissolved in distilled water to the original volume and precipitate with ethanol again (2:1, v/v, ethanol/water). Collected precipitates were dried under vacuum at room temperature. After drying, the product was redissolved in distilled water, filtered and then freeze dried under reduced pressure.

A polyester triol(Mw=900 Da) was synthesized from a glycerol starter with a backbone comprising 70 wt % ε-caprolactone, 20 wt % glycolide, and 10 wt % D,L-lactide [12]. Briefly, to make a 80 g batch of the polyester triol, 8.186 g dried glycerol, 50.270 g dried ε-caprolactone, 14.363 g glycolide, 7.181 g D,L-lactide, and 0.1 wt % catalyst (0.8 g stannous octoate) were mixed in a 100 mL reaction flask with mechanical stirring under argon gas flow for 36 h at 140° C. The product was washed with hexane for three times and then dried under vacuum at 80° C. for 48 h. A high isocyanate (NCO) LTI-PEG prepolymer (21,000 cP, NCO:OH equivalent ratio=3.0:1.0) was synthesized by adding PEG (Mw=200 g/mol) dropwise to LTI in a 100 mL reaction flask with mechanical stirring under argon gas flow for 24 h at 45° C. [13]. The prepolymer was then dried under vacuum at 80° C. overnight.

Carbon dioxide foaming polyurethane scaffolds were synthesized by reactive liquid molding of the prepolymer with a hardener polyol. The hardener polyol here was prepared by mixing 100 parts the polyester triol with alginate beads and 10.0 pphp (parts per hundred parts) iron catalyst (5% iron acetylacetonate solution in 2,3-Diphosphoglycerate). A Hauschild SpeedMixer™ DAC 150 FVZ-K vortex mixer (FlackTeck, Inc., Landrum, S.C.) was used for the mixing process.

The scaffold pore size distribution and its internal pore morphology were determined by scanning electron microscopy (Hitachi S-4200 SEM, Finchampstead, UK) with gold sputter coating with a Cressington Sputter Coater. Samples were dried under vacuum at room temperature for 48 h and sliced before gold coating. Porosities were calculated from mass and volume measurements of cylindrical scaffold cores after drying under vacuum for 48 h at room temperature. The density of polyurethane used for calculation was 1200 kg·m^{-3 [}14].

Young's modulus from submersion compression measured by a TA Instruments Q800 Dynamic Mechanical Analyzer (DMA) was used to describe mechanical properties of wet scaffolds. Samples were tested when kept overnight after fabrication. Stress-strain curves were generated by compressing cylindrical shaped samples at 37° C. under water at a rate of 0.1 N/min and Young's modulus was determined from the slope of the linear region of the curve.

To investigate if the encapsulation process would harm the cells, live/dead staining (Live/Dead® Viability/Cytotoxicity Kit for mammalian cells, invitrogen) was used to examine cell viability after alginate hydrogel and polyurethane formation. Alginate beads with cells were harvested from the gelling agent solution or polyurethane scaffold. After 30 min staining, fluorescent microscope (Olympus) was used to capture images with live/dead cells inside beads. The viability was calculated by: Viability=Nlive/(Nlive=Ndead), where Nlive=the number of live cells (green), and Ndead=the number of dead cells (red) [15].

Results

For the electronic bead maker used in this Example, alginate bead size could be reduced by decreasing diameter of the nozzle, decreasing flow rate of the pumped sodium alginate solution and increasing applied voltage [10]. At the same time, as described above, smaller bead size was preferred in this study, while high voltage may hurt cells. Therefore, in order to gain smaller beads with lower applied voltage, small nozzle diameter of 0.35 mm and low sodium alginate flow rate of 10 mL/h was selected.

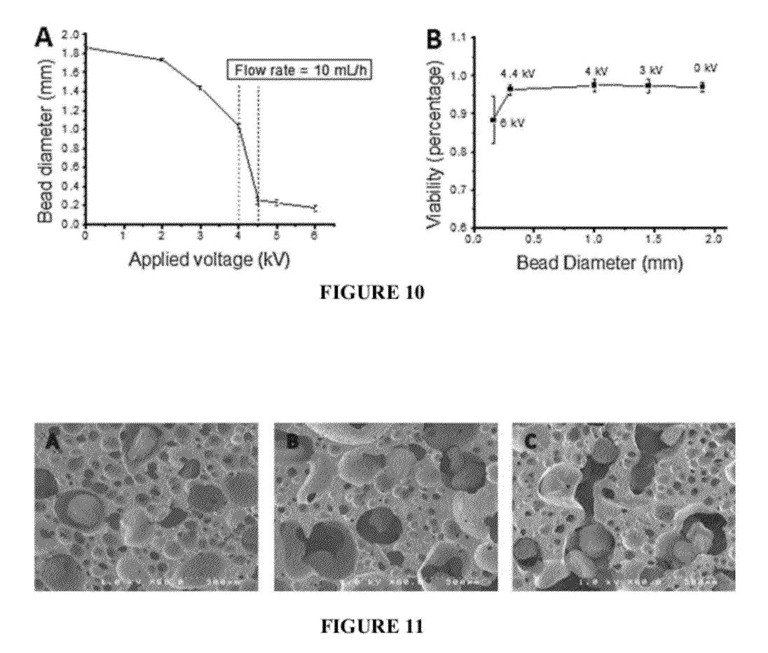

FIG. 10 (A) shows the effect of applied voltage on alginate bead diameter, indicating that increasing the applied voltage leads to reducing the bead size to some minimum value [10]. The trend of the curve reveals that bead size reduced relatively low at the early stages of voltage increase (below 4 kV), while the reduction speeded up from 4 kV to 4.5 kV and then slowly went to a minimum.

Since bead size here was controlled by potential difference, which could do harm to cells, cell viabilities of different bead sizes were compared after alginate encapsulation process. FIG. 10 (B) shows the viability (percentage of live cells) trend with the reduction of bead diameter. 300 μm (4.4 kV) was selected as the average alginate bead diameter for next experiments in this study.

In order to give consideration to both the amount of encapsulated cells and mechanical properties of polyurethane scaffold, different bead loadings were compared. SEM images (FIG. 11) of polyurethane scaffold with alginate beads indicates two distributions of inter pore sizes. See Table 2, below.

Big pores (˜270 μm) were occupied by alginate beads, while micro pores (˜60 μm) were caused by carbon dioxide foaming during the formation of polyurethane. Moreover, SEM images also proved that those bead occupied pores were interconnected with the micro pores, which is important for supporting cells with nutrients and oxygen inside polyurethane. Since FIG. 11 (C) (60% loading) shows obvious defects inside polyurethane, only 40% and 50% loadings were kept for further comparison. Table 2 reveals that although 50% loading could provide more bead occupied pores, the fraction of micro pores decreased, yielding a similar pore fraction with 40% loading. Porosities calculated from mass and volume measurements also showed consistent results for 40% (˜71.7%) and 50% (˜71.5%) loadings. Young's modulus of submersion compression, which represented mechanical properties of polyurethane scaffolds, is also compared in Table 2. Both measured values are consistent with reported modulus of polyurethane scaffolds [16], while 40% loading showed to be better than 50% loading. Yet, both moduli are at the same order of magnitude, meaning not much difference, and in order to convey more cells, 50% loading was selected for cell study.

TABLE 2
Characterization of inter pores and modulus of polyurethane scaffold.

| Bead | Bead pore | Micro pore | Bead pore | Micro pore | Pore area | Modulus |
|---|---|---|---|---|---|---|
| Loading | size (μm) | size (μm) | area (%) | area (%) | (%) | (MPa) |
| 40% | 270.2 | 67.9 | 36.43 | 30.15 | 66.58 | 0.217 ± 0.027 |
| 50% | 261.64 | 52.13 | 50.79 | 16.76 | 67.55 | 0.132 ± 0.036 |

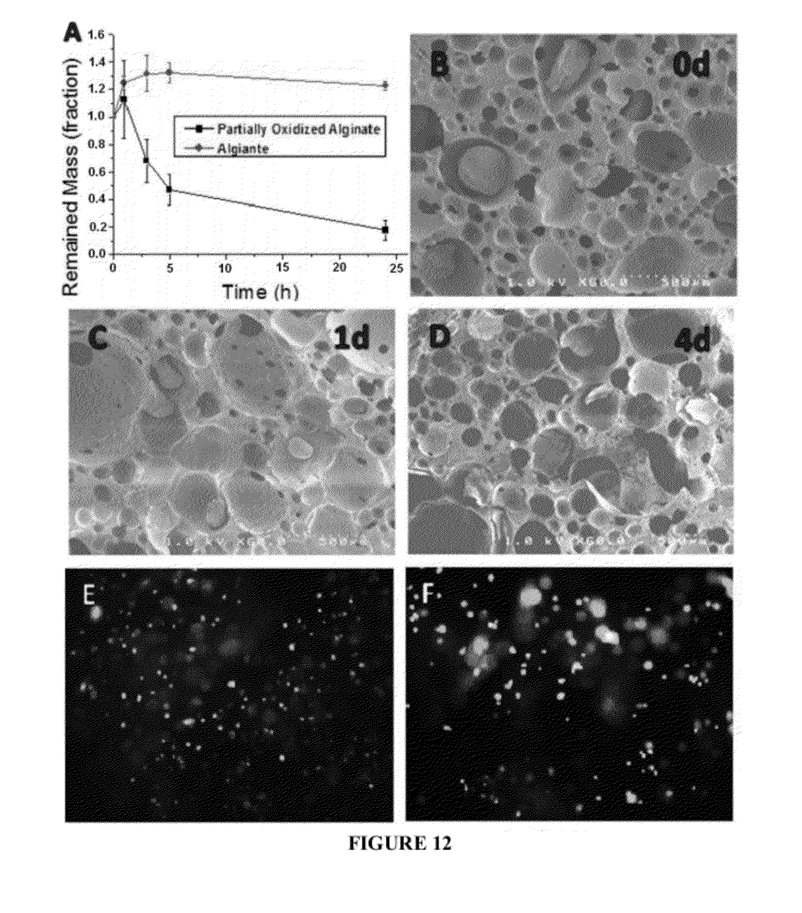

Sodium alginate was partially oxidized to a theoretical extent of 5%. Partially oxidized alginate may not significantly interfere in the formation of ionic junctions with divalent cations [11]. Degradation rate of oxidized alginate hydrogel was markedly faster than normal alginate (FIG. 3 (A)). For 24 h culturing in complete medium in shaker, only 20% oxidized hydrogel mass was left, while normal alginate hydrogel swelled a lot. Because of the limited diffusion, the degradation rate decreased inside polyurethane scaffold. SEM images (FIG. 12 (B) (C) (D)) showed that after culturing the scaffolds with oxidized alginate hydrogel under the same condition for 4 days, almost all the hydrogel were degraded and diffused away. These results clearly demonstrate that oxidized alginate hydrogels degrade over a relatively short period. At the same time, considering the excellent biocompatibility might be changed due to the modification process, cell viability encapsulated in partially oxidized alginate hydrogel was determined. FIGS. 12 (E) and (F) suggest no effect of the oxidation process on cell viability.

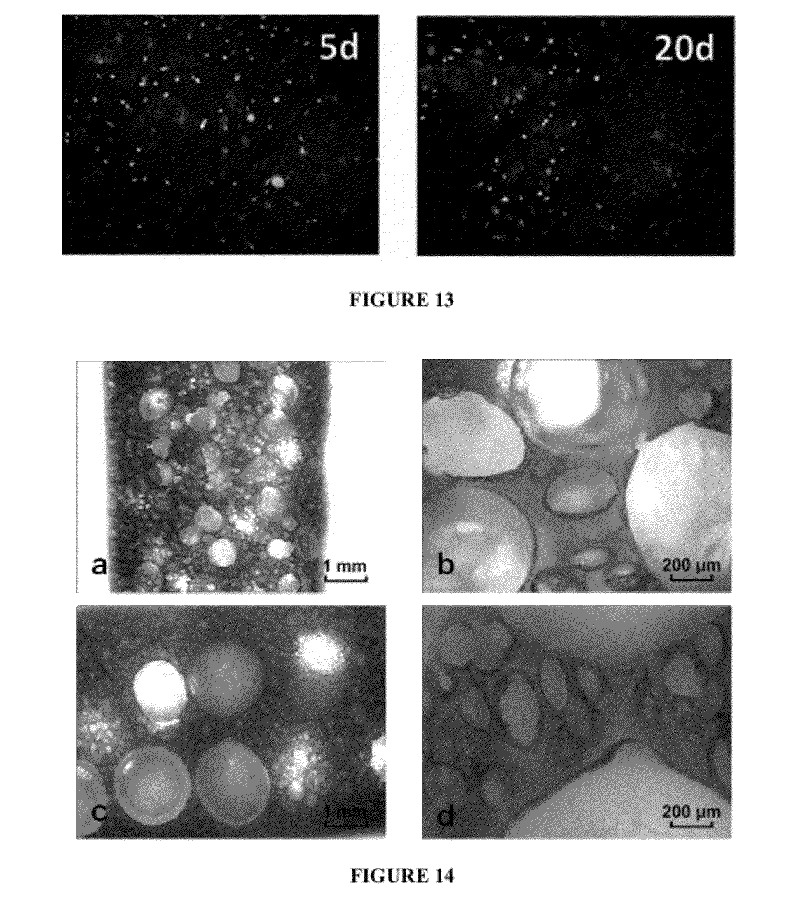

As mentioned above, the formation process of polyurethane is harmful for cell survivability. Even though protected by alginate hydrogel, cell viability after the process was determined. FIG. 13 shows live/dead staining of cells encapsulated in polyurethane (bead diameter=2.6 mm) after culturing for 5 and 20 days. Over 80% cells survived for up to 20 days. This result suggests that cells were able to survive long term inside polyurethane scaffolds, proving the possibility of polyurethane as a cell carrier for in situ tissue repair.

### Example 5

This Example also relates to further embodiments of PUR composites comprising cells, including cells encapsulated in alginate beads.

Materials & Methods: Similarly to Example 4, injectable polyurethanes synthesized from a polyester triol, an iron catalyst, and a lysine triisocyanate-PEG (LTI-PEG) prepolymer were used as carriers for cell delivery. Considering that the reactants of polyurethane are highly reactive, cells cannot be encapsulated directly due to the chemical reaction between NCO-terminated prepolymer and the cells. In order to protect cells during the process of curing, we encapsulated MC3T3-E1 cells in calcium alginate hydrogel beads with diameter ranging from 300-800 μm. Cell viability was assessed using a Live/Dead viability kit (Invitrogen). In order to increase the degradation rate of the alginate, which was designed to protect the cells from chemical reaction and subsequently degrade over 1-2 days, we investigated oxidized alginate for encapsulating cells. The degradation of alginate beads embedded in the scaffolds was evaluated by SEM.

Results & Discussion: Cells were encapsulated in alginate beads with high viability (e.g., >90%). When 50 wt % alginate beads were embedded in the reactive polyurethane scaffolds, SEM images revealed the presence of both the beads and interconnected micro pores of about 50-70 μm (see e.g., FIG. 11), resulting in a percolated pathway for transport of oxygen, fluid, and nutrients into the interior of the scaffold. Interconnectivity decreased substantially when 40 wt % beads were added, as evidenced by minimal infiltration of medium into the interior of the scaffold. For 60 wt % beads, the scaffolds had large voids and were friable.

In order to release cells after final cure of the scaffold, partially oxidized sodium alginate was used for encapsulation. Cell viability was unchanged when encapsulated in the partially oxidized alginate. SEM images revealed that the partially oxidized alginate embedded within the polyurethane scaffold degraded after 3-4 days in dynamic culture, thereby creating pores within the scaffold (FIG. 12).

Viable (>80%) cells were observed in the slowly degrading alginate beads embedded in the polyurethane scaffolds for up to 20 days in vitro (bead size: 2 mm). However, without being bound by theory or mechanism, cell viability may decrease with decreasing bead diameter due to temperature or CO₂ concentration gradients generated by the exothermic reaction. Tests have shown that unreacted components extract from the polyurethane scaffolds are not cytotoxic.

### Example 6

This Example relates to local cell delivery from injectable biodegradable polymeric scaffolds.

Materials and Methods

MC3T3-E1 mouse osteoblast precursor cells were used in this study. Cells were cultured in α-minimum essential medium (α-MEM) with 10% (v/v) fetal bovine serum (FBS) and 1% (v/v) penicillin (100 U/ml)/streptomycin (100 μg/ml) in a CO₂ incubator with 5% CO₂ at 37° C. The culture medium was changed every 2 days. Trypsin-EDTA was used to recover MC3T3-E1 cells.

Alginate was used as an encapsulating gel to protect the cells during the polymerization reaction of polyurethane (PUR). Alginate is bio compatible and forms a cross-linked gel under mild conditions. Alginic acid sodium salt (viscosity 20,000˜40,000 cps, Aldrich) was dissolved in DI-water and in α-MEM at concentrations of 1 and 2% (w/v). Briefly, MC3T3-E1 cells (1×10⁶ cells/ml) were suspended in the alginic acid sodium salt solutions (1 and 2% (w/v)) and mixed for 2 h. Suspensions of cells in alginate were then added drop-wise into two different crosslinker solutions: (a) CaCl₂ in DI-water or (b) CaCl₂ in α-MEM at room temperature. The concentration of CaCl₂ solutions was either 100 or 200 mM. The beads formed in the microencapsulation device were subsequently cured in the CaCl₂ solution for 1 h.

Polyester triols (900 Da) were prepared from a glycerol starter and a backbone comprising 70 wt % 3-caprolactone, 20 wt % glycolide, and 10 wt % D,L-lactide as published previously in Refs. [1, 2]. A high isocyanate (NCO) LTI-PEG prepolymer (21,000 cP, NCO:OH equivalent ratio=3.0:1.0) was synthesized by adding PEG (Mw=200 g/mol) dropwise to LTI in a 100 mL reaction flask with mechanical stirring under argon gas flow for 24 h at 45° C. The prepolymer was then dried under vacuum at 80° C. overnight.

PUR scaffolds were synthesized by one-shot reactive liquid molding of the prepolymer with a hardener polyol comprising 100 parts the polyester triol and 10.0 pphp (parts per hundred parts) iron catalyst (5 wt. % in 2,3-Diphosphoglycerate). 50 wt % alginate beads per total weight of the scaffold were added to the hardener component before mixing with the prepolymer and mixed for 30 seconds in A Hauschild SpeedMixer™ DAC 150 FVZ-K vortex mixer (FlackTeck, Inc., Landrum, S.C.). The prepolymer was added to the resulting mixture and mixed for 30 seconds in the mixer. The final mixture injects into a cylindrical mold and cured for 10 min.

Optical microscopy (Olympus CKX41) and scanning electron microscopy (Hitachi S-4200 SEM, Finchampstead, UK) were utilized to measure the pore size and determine the internal pore morphology of the alginate/PUR scaffolds.

4 mM cell-permeable calcein acetoxymethyl (Calcein AM) and 2 mM ethidium homodimer-1 (EthD-1) from the Live/Dead Viability/Cytotoxicity Kit (Invitrogen) for mammalian cells was added to the samples. Calcein AM produces a bright green fluorescence in live cells. Ethidium homodimer-1 is retained within damaged or dead cells, imparting a bright red fluorescence. Fluorescence images were observed by an Olympus DP71 camera attached to a fluorescent microscope (Olympus CKX41, U-RFLT50, Center Valley, Pa.). Cell viability (the percentage of viable cells) was measured by counting the numbers of live and dead cells from the fluorescent images at time points of 0, 1, 2 and 5 days.

Alginate beads were harvested from the alginate/PUR scaffold (500 mg) in 24-well plates at every time points and immersed in phosphate-buffered saline (PBS) for 5 min followed by washing with PBS 3 times. The washed beads were crushed and lysed with 150 ml of 0.05% Triton X-100. The plates were homogenized by three freeze-thaw cycles. The lysates (20 ml) were added to 96-well plates with 100 ml substrate buffer (2 mg/ml disodium p-nitrophenylphosphate hexahydrate and 0.75 M 2-amino-2-methyl-1-propanol). The mixtures were then incubated for 30 min at 37° C. and the resulting optical absorbance measured at 410 nm by using a mQuant spectrophotometer (Bio-Tek Instruments Inc.). ALP activity was determined from a standard curve generated by employing the reaction of a p-nitrophenyl solution. The ALP activity was normalized using the measured total protein to account for differences in the number of cells on different alginate beads at individual time points. Total cellular protein was determined with a BCA protein assay kit (Pierce). The lysates (10 ml) were mixed with 200 ml BCA working reagent containing cupric sulfate and bicinchoninic acid in 96-well plates, and then incubated for 30 min at 37° C. The resulting optical absorbance was measured at 562 nm with the μQuant spectrophotometer. Total protein amounts were calculated with a standard curve, which was generated with bovine serum albumin.

Results

The synthesized Ca-alginate/PUR composite exhibited three different pore structures: macropores (0.5-2 mm) from degradation of alginate beads, intermediate pores (200 μm), and micropores (several μm) in the PUR matrix (FIG. 14).

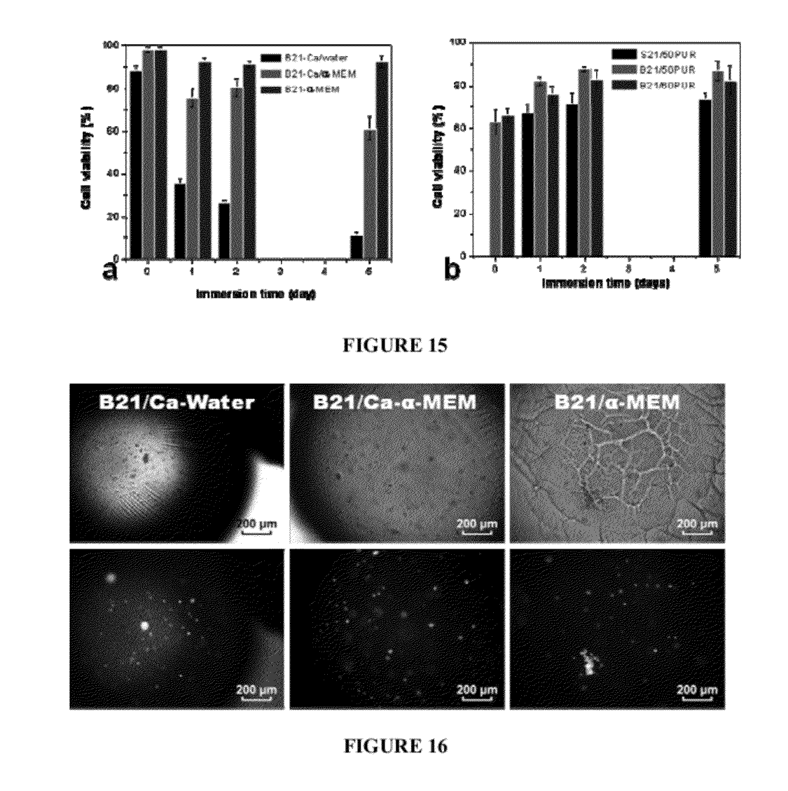

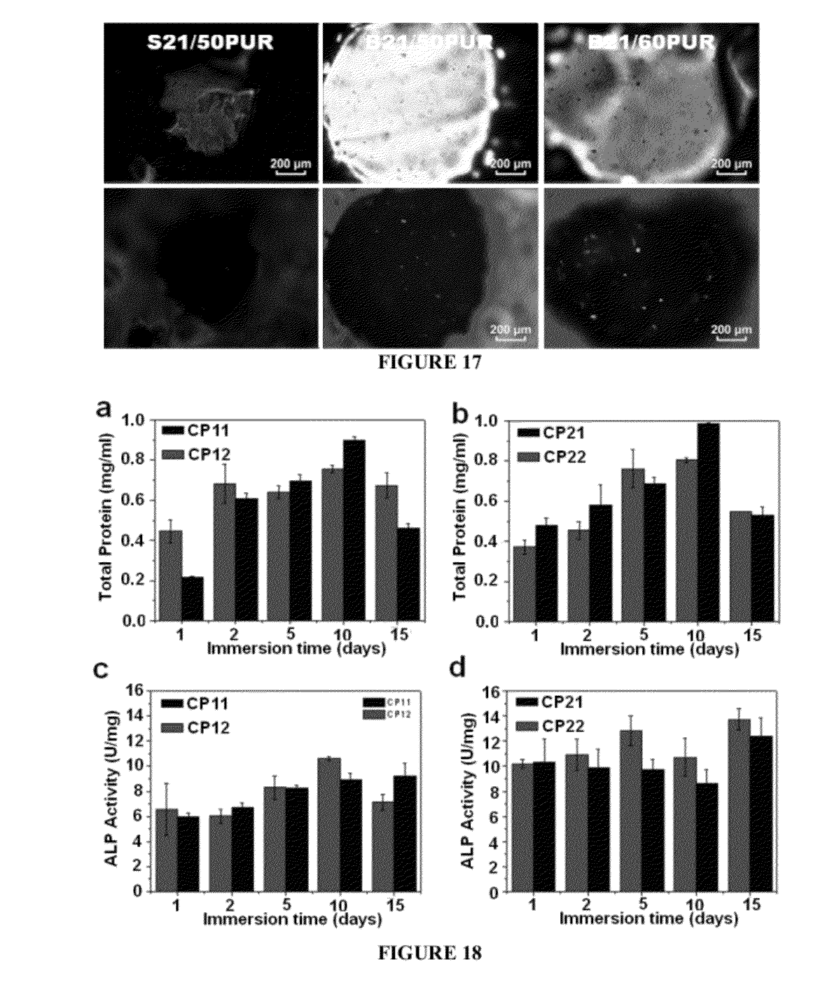

encapsulating MC3T3-E1 cells in α-MEM alginate solution protects the cells from the polymerization and improves cell viability (FIG. 15-17). Cell encapsulations within 3D alginate hydrogel matrix were performed with two kinds of solvents, which were DI-water and α-MEM, for alginic acid solution and CaCl₂ solution. As representatively shown in FIG. 14(a), almost encapsulated cells were well survived with α-MEM solvents during the synthesis process in the alginate beads, which is 2 mm diameter reacting 2 wt. % alginic acid and 100 mM CaCl₂ solutions. However, 15% of cell death was observed in the synthesized beads with DI-water solvents. Green fluorescence means a live cell and red fluorescence means a dead cell in LIVE/DEAD image. The cell viability of Ca-alginate beads with α-MEM (>99%) was significantly higher than that of Ca-alginate beads formed with DI-water (<85) after cell encapsulation (FIG. 14(a)). After 5 day soaking in α-MEM/CaCl₂ solution, cells were survived over 60% in the alginate beads even if the solution included a lot of CaCl₂. It is conjectured that using α-MEM as the solvents for polymer matrix and catalyst solutions to prepare the alginate beads supplied nutrients and oxygen during cell encapsulation and incorporating into PUR scaffold. As shown in FIG. 17, the encapsulated cells in alginate showed good viability in all specimens even if the beads were incorporated into PUR matrix. In representative OM and live/dead (L/D) staining images of the alginate S21 alginate beads (φ=1 mm), few dead encapsulated MC3T3-E1 cells (red fluorescent) were shown in the edge of beads (FIGS. 17-18). However, almost cells (green fluorescent) were survived in B21 beads (φ=2 mm). The injectable alginate/PUR composite scaffold also supports differentiation of MC3T3-E1 preosteoblast cells (FIG. 18). It is conjectured that α-MEM in alginate beads supplies nutrients and oxygen for the encapsulated cells during PUR polymerization, and that connectivity between Ca-alginate beads facilitates transport of nutrients after polymerization.

### Example 7

This Example relates to controlling gel bead size by varying applied voltage, the effects of applied voltage on cell viability, and the effect of oxidizing alginate bead on degradation rate in α-MEM.

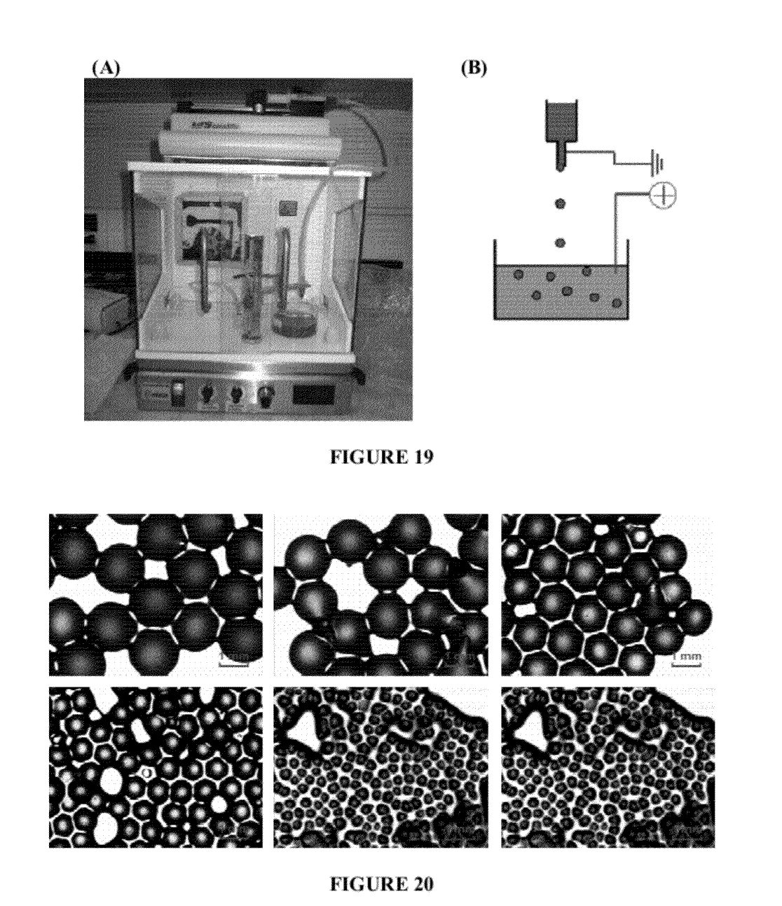

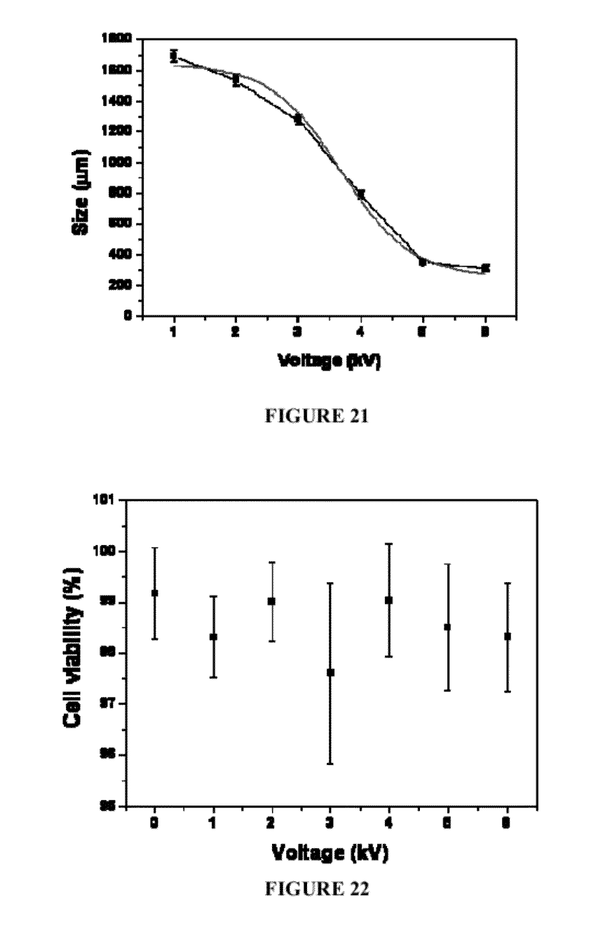

Alginate beads were formed using alginic acid and its sodium salt (sigma; 20,000˜40,000 cps) at 2% (w/v) in α-MEM and a cross-linking agent comprising of 100 mM CaCl₂ in α-MEM as discussed in the previous Examples. Gelling was allow to proceed for 2 h and 1-6 kV were applied using the Var-V1 encapsulation unit (FIG. 19). Using this method and system, alginate beads were successfully synthesized of 300˜1700 μm, and size was controlled by varying voltage (FIGS. 20-21).

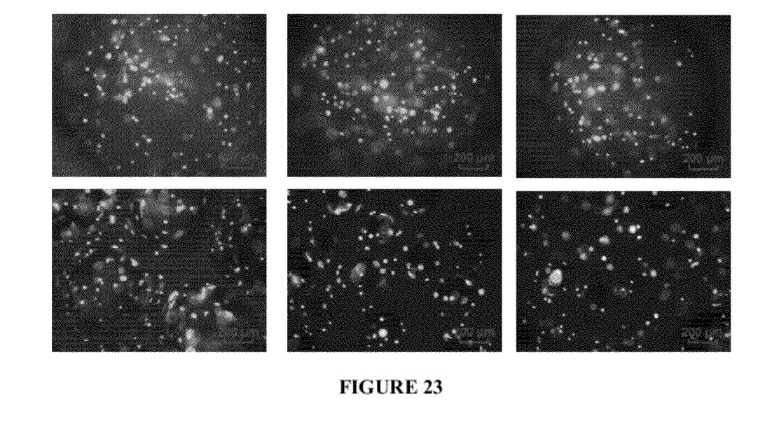

MC3T3-E1 cells at 1×10⁶ cells/ml were encapsulated using the same system and method. Cells showed high viability (e.g. greater than 95%) for all voltages tested (FIGS. 22-23).

Lastly, the alginate used for the cell encapsulation was oxidized as described in Example 4. The stoichiometric ratio of sodium priodate to alginate was adjusted so as to achieve a theoretical oxidation of 7.5% of the alginate. Beads were then formed as discussed above in this Example, but using cell encapsulated in the oxidized alginate. Subsequent degradation studies showed that 2-200 and 2-100 embodiments degraded at approximately the same rate, and degraded by over 80% after 2 days of immersion in α-MEM. On the other hand, 1-200 alginate beads that were not oxidized took were only about 60% degraded after 10 days of immersion in α-MEM. It was further noted that 1-200 beads made from oxidized alginate degraded at a slower rate than then 2-100 and 2-200 embodiments, notwithstanding them having a smaller bead size (e.g. less than 0.2 mm) and a lower concentration of alginate. Thus, encapsulating cells in partially oxidized alginate was shown to be a suitable method for accelerating alginate bead degradation.

Throughout this application, various publications are referenced. All such references, including the follow listed references, are incorporated herein by reference.

### REFERENCES

[1] Hernandez R M, Orive G, Murua A, Pedraz J L. Microcapsules and microcarriers for in situ cell delivery. Adv Drug Deliv Rev 2010; 62:711-30.
[2] Zhang W, Tucker-Kellogg L, Narmada B C, Venkatraman L, Chang S, Lu Y, et al. Cell-delivery therapeutics for liver regeneration. Adv Drug Deliv Rev 2010; 62:814-26.
[3] Mandal B B, Kundu S C. Calcium alginate beads embedded in silk fibroin as 3D dual drug releasing scaffolds. Biomaterials 2009; 30:5170-7.
[4] Zhou H, Xu H H. The fast release of stem cells from alginate-fibrin microbeads in injectable scaffolds for bone tissue engineering. Biomaterials 2011; 32:7503-13.
[5] Wang C, Gong Y, Lin Y, Shen J, Wang D A. A novel gellan gel-based microcarrier for anchorage-dependent cell delivery. Acta Biomater 2008; 4:1226-34.
[6] Wagers A J, Weissman I L. Plasticity of adult stem cells. Cell 2004; 116:639-48.
[7] Mooney D J, Vandenburgh H. Cell delivery mechanisms for tissue repair. Cell Stem Cell 2008; 2:205-13.
[8] Hofmann M, Wollert K C, Meyer G P, Menke A, Arseniev L, Hertenstein B, et al. Monitoring of bone marrow cell homing into the infarcted human myocardium. Circulation 2005; 111:2198-202.
[9] Evans S M, Mummery C, Doevendans P A. Progenitor cells for cardiac repair. Seminars in cell & developmental biology 2007; 18:153-60.
[10] Orive G, De Castro M, Kong H J, Hernandez R M, Ponce S, Mooney D J, et al. Bioactive cell-hydrogel microcapsules for cell-based drug delivery. J Control Release 2009; 135:203-10.
[11] Wang N, Adams G, Buttery L, Falcone F H, Stolnik S Alginate encapsulation technology supports embryonic stem cells differentiation into insulin-producing cells. J Biotechnol 2009; 144:304-12.
[12] Wikstrom J, Elomaa M, Syvajarvi H, Kuokkanen J, Yliperttula M, Honkakoski P, et al. Alginate-based microencapsulation of retinal pigment epithelial cell line for cell therapy. Biomaterials 2008; 29:869-76.
[13] Chen C S, Mrksich M, Huang S, Whitesides G M, Ingber D E. Geometric control of cell life and death. Science 1997; 276:1425-8.
[14] Burdick J A, Vunjak-Novakovic G. Engineered microenvironments for controlled stem cell differentiation. Tissue Eng Part A 2009; 15:205-19.
[15] Singhvi R, Kumar A, Lopez G P, Stephanopoulos G N, Wang D I, Whitesides G M, et al. Engineering cell shape and function. Science 1994; 264:696-8.
[16] Lim J Y, Donahue H J. Cell sensing and response to micro- and nanostructured surfaces produced by chemical and topographic patterning. Tissue Eng 2007; 13:1879-91.
[17] Fuhrmann T, Hillen L M, Montzka K, Woltje M, Brook G A. Cell-cell interactions of human neural progenitor-derived astrocytes within a microstructured 3D-scaffold. Biomaterials 2010; 31:7705-15.
[18] Kuppers M, Faust D, Linz B, Dietrich C. Regulation of ERK1/2 activity upon contact inhibition in fibroblasts. Biochem Biophys Res Commun 2011; 406:483-7.
[19] Eagle H, Levine E M. Growth regulatory effects of cellular interaction. Nature 1967; 213:1102-6.
[20] Zeng Q, Hong W. The emerging role of the hippo pathway in cell contact inhibition, organ size control, and cancer development in mammals. Cancer Cell 2008; 13:188-92.
[21] Chan A W, Neufeld R J. Tuneable semi-synthetic network alginate for absorptive encapsulation and controlled release of protein therapeutics. Biomaterials 2010; 31:9040-7.
[22] Bouhadir K H, Lee K Y, Alsberg E, Damm K L, Anderson K W, Mooney D J. Degradation of partially oxidized alginate and its potential application for tissue engineering. Biotechnol Prog 2001; 17:945-50.
[23] Zhao L, Weir M D, Xu H H. An injectable calcium phosphate-alginate hydrogel-umbilical cord mesenchymal stem cell paste for bone tissue engineering. Biomaterials 2010; 31:6502-10.
[24] Jeon O, Bouhadir K H, Mansour J M, Alsberg E. Photocrosslinked alginate hydrogels with tunable biodegradation rates and mechanical properties. Biomaterials 2009; 30:2724-34.
[25] Rowley J A, Madlambayan G, Mooney D J. Alginate hydrogels as synthetic extracellular matrix materials. Biomaterials 1999; 20:45-53.
[26] Evangelista M B, Hsiong S X, Fernandes R, Sampaio P, Kong H J, Barrias C C, et al. Upregulation of bone cell differentiation through immobilization within a synthetic extracellular matrix. Biomaterials 2007; 28:3644-55.
[27] Augst A D, Kong H J, Mooney D J. Alginate hydrogels as biomaterials. Macromol Biosci 2006; 6:623-33.
[28] Rowley J A, Mooney D J. Alginate type and RGD density control myoblast phenotype. J Biomed Mater Res 2002; 60:217-23.
[29] Comisar W A, Kazmers N H, Mooney D J, Linderman J J. Engineering RGD nanopatterned hydrogels to control preosteoblast behavior: a combined computational and experimental approach. Biomaterials 2007; 28:4409-17.
[30] Fonseca K B, Bidarra S J, Oliveira M J, Granja P L, Barrias C C. Molecularly designed alginate hydrogels susceptible to local proteolysis as three-dimensional cellular microenvironments. Acta Biomater 2011; 7:1674-82.
[31] Jeon 0, Powell C, Ahmed S M, Alsberg E. Biodegradable, photocrosslinked alginate hydrogels with independently tailorable physical properties and cell adhesivity. Tissue Eng Part A 2010; 16:2915-25.
[32] Zimmermann U, Thurmer F, Jork A, Weber M, Mimietz S, Hillgartner M, et al. A novel class of amitogenic alginate microcapsules for long-term immunoisolated transplantation Ann N Y Acad Sci 2001; 944:199-215.
[33] Discher D E, Janmey P, Wang Y L. Tissue cells feel and respond to the stiffness of their substrate. Science 2005; 310:1139-43.
[34] Choi B G, Park M H, Cho S H, Joo M K, Oh H J, Kim E H, et al. In situ thermal gelling polypeptide for chondrocytes 3D culture. Biomaterials 2010; 31:9266-72.
[35] Engler A J, Sen S, Sweeney H L, Discher D E. Matrix Elasticity Directs Stem Cell Lineage Specification. Cell 2006; 126:677-89.
[36] Kong H J, Liu J, Riddle K, Matsumoto T, Leach K, Mooney D J. Non-viral gene delivery regulated by stiffness of cell adhesion substrates. Nature materials 2005; 4:460-4.
[37] Taqvi S, Roy K. Influence of scaffold physical properties and stromal cell coculture on hematopoietic differentiation of mouse embryonic stem cells. Biomaterials 2006; 27:6024-31.
[38] Gilbert P M, Havenstrite K L, Magnusson K E, Sacco A, Leonardi N A, Kraft P, et al. Substrate elasticity regulates skeletal muscle stem cell self-renewal in culture. Science 2010; 329:1078-81.
[39] Ruppender N S, Merkel A R, Martin T J, Mundy G R, Sterling J A, Guelcher S A. Matrix rigidity induces osteolytic gene expression of metastatic breast cancer cells. PLoS ONE 2010; 5:e15451.
[40] Sperling L H. Introduction to Physical Polymer Science. 3rd ed. New York: Wiley-Interscience; 2001.
[41] Raeber G P, Lutolf M P, Hubbell J A. Molecularly engineered PEG hydrogels: a novel model system for proteolytically mediated cell migration. Biophys J 2005; 89:1374-88.
[42] Smidsrod O, Soution properties of alginate. Carbohydr Res 1970; 13:359-72.
[43] Chan A W, Neufeld R J. Modeling the controllable pH-responsive swelling and pore size of networked alginate based biomaterials. Biomaterials 2009; 30:6119-29.
[44] Caykara T, Inam R. Determination of Average Molecular Weight Between Crosslinks and Polymer-Solvent Interaction Parameters of Poly(acrylamide-g-ethylene diamine tetraacetic acid) Polyelectrolyte Hydrogels. J Appl Polym Sci 2004; 91:2168-75.
[45] Amsden B, Turner N. Diffusion characteristics of calcium alginate gels. Biotechnol Bioeng 1999; 65:605-10.
[46] Moe S T, Draget K I, Skjak-Braek G, Smidsrod 0. Temperature dependence of the elastic modulus of alginate gels. Carbohydr Polym 1992; 19:279-84.
[47] Jeon O, Song S J, Lee K-J, Park M H, Lee S—H, Hahn S K, et al. Mechanical properties and degradation behaviors of hyaluronic acid hydrogels cross-linked at various cross-linking densities. Carbohydr Polym 2007; 70:251-7.
[48] Lee K Y, Mooney D J. Alginate: properties and biomedical applications. Prog Polym Sci 2010.
[49] Davidovich-Pinhas M, Bianco-Peled H. A quantitative analysis of alginate swelling. Carbohydr Polym 2010; 79:1020-7.
[50] Kong H J, Lee K Y, Mooney D J. A quantitative analysis of alginate swelling. Macromolecules 2003; 36:7887-90.
[51] Martinsen A, Storro I, Skjark-Braek G. Alginate as immobilization material: III. Diffusional properties. Biotechnol Bioeng 1992; 39:186-94.
[52] West E R, Xu M, Woodruff T K, Shea L D. Physical properties of alginate hydrogels and their effects on in vitro follicle development. Biomaterials 2007; 28:4439-48.
[53] Abbah S A, Lu W W, Chan D, Cheung K M, Liu W G, Zhao F, et al. Osteogenic behavior of alginate encapsulated bone marrow stromal cells: an in vitro study. J Mater Sci Mater Med 2008; 19:2113-9.
[1] Chunmeng Shi, Ying, Zhu, Yongping Su, Tianmin Cheng; Stem cells and their applications in skin-cell therapy; Trends in Biotechnology, Vol. 24 No. 1, January 2006.
[2] Anthony D. Metcalfe, Mark W. J. Ferguson; Tissue engineering of replacement skin: the crossroads of biomaterials, wound healing, embryonic development, stem cells and regeneration; Journal of the royal society interface (2007) 4, 413-437.
[3] Ellen Heber-Katz, John Leferovich, Khamilia Bedelbaeva, Dmitri Gourevitch, Lise Clark; The scarless heart and the MRL mouse; The Royal Society online 8 Apr. 2004.
[4] David J. Mooney, Herman Vandenburgh; Cell Delivery Mechanisms for Tissue Repair; Cell Stem Cell 2, March 2008.
[5] Bing Li, Jeffrey M. Davidson, Scott A. Guelcher; The effect of the local delivery of platelet-derived growth factor from reactive two-component polyurethane scaffolds on the healing in rat skin excisional wounds; Biomaterials 30 (2009) 3486-3494.
[6] Bing Li, Toshitaka Yoshii, Andrea E. Hafeman, Jeffry S, Nyman, Joseph C. Wenke, Scott A. Guelcher; The effects of rhBMP-2 released from biodegradable polyurethane/microsphere composite scaffolds on new bone formation in rat femor; Biomaterials 30 (2009) 6768-6779.
[7] Rosa M Hernandez, Gorka Orive, Ainhoa Murua, Jose Luis Pedraz; Microcapsules and microcarriers for in situ cell delivery; Advanced Drug Delivery Reviews (2010).
[8] Nan Wang, Gary Adams, Lee Buttery, Franco H. Falcone, Snow Stolnik; Alginate encapsulation technology supports embryonic stem cells differentiation into insulin-producing cells; Journal of Biotechnology 144 (2009) 304-312.
[9] Liang Zhao, Michael D. Weir, Hockin H. K. Xu; An injectable calcium phosphate-algiante hydrogel-umbilical cord mesenchymal stem cell paste for bone tissue engineering; Biomaterials 31 (2010) 6502-6510.
[10] Hamid Moghadam, Mohsen Samimi, Abdolreza Samimi, Mohamad Khorram; Electro-spray of high viscous liquids for producing mono-sized spherical alginate beads; Particuology 6 (2008) 271-275.
[11] Kamal H. Bouhadir, Kuen Yong Lee, Eben Alsberg, Kelly L. Damm, Kenneth W. Anderson, David J. Mooney; Degradation of Partially Oxidized Alginate and Its Potential Application for Tissue Engineering; Biotechnol. Prog. 2001, 17, 945-950.
[12] Scott A. Guelcher, Vishal Patel, Katie M. Gallagher, Susan Connolly, Jonathan E. Didier, John S. Doctor, Jeffery 0. Hollinger; Synthesis and In Vitro Biocompatibility of Injectable Polyurethane Foam Scaffolds; Tissue Engineering, Volume 12, Number 5, 2006.
[13] Jerold E. Dumas, Katarzyna Zienkiewics, Shaun A. Tanner, Edna M. Prieto, Subha Bhattacharyya, Scott A. Guelcher; Synthesis and Characterization of an Injectable Allograft Bone/Polymer Composite Bone Void Filler with Tunable Mechanical Properties; Tissue Engineering: Part A, Volume 16, Number 8, 2010.
[14] Bayer Material Science. Desmopan 385E ISO Datasheet. Pittsburgh, Jul. 7, 2004.
[15] Liang Zhao, Michael D. Weir, Hockin H. K. Xu; An injectable calcium phosphate-algiante hydrogel-umbilical cord mesenchymal stem cell paste for bone tissue engineering; Biomaterials 31 (2010) 6502-6510.
[16] Scott A. Guelcher; Biodegradable Polyurethanes: Synthesis and Applications in Regenerative Medicine; Tissue Engineering: Part B, Volume 14, Number 1, 2008.

What is claimed is:

1. A biodegradable composite, comprising:
a polyurethane component; and
cells encapsulated in gel beads.

2. The composite of claim 1, wherein the gel beads have a size of about 200 μm to about 300 μm.

3. The composite of claim 1, wherein the gel beads have a size of about 300 μm to about 800 μm.

4. The composite of claim 1, wherein the gel beads have a size of about 800 μm to about 2 mm.

5. The composite of claim 1, wherein the gel beads further comprise a formulation for culturing cells.

6. The composite of claim 5, wherein the formulation for culturing cells is selected from the group consisting of α-MEM, deionized water, PBS, DMEM, and combinations thereof.

7. The composite of claim 1, wherein the gel beads are alginate beads.

8. The composite of claim 7, wherein the alginate beads are formed from at least sodium alginate and a calcium catalyst.

9. The composite of claim 8, wherein the sodium alginate has a concentration of about 1% to about 2% (w/v).

10. The composite of claim 8, wherein the calcium catalyst has a concentration of about 100 mM to about 200 mM.

11. The composite of claim 8, wherein the calcium catalyst is CaCl₂.

12. The composite of claim 7, wherein the alginate beads are partially oxidized.

13. The composite of claim 12, wherein the alginate beads are oxidized by about 0.1% to about 10%.

14. The composite of claim 13, wherein the alginate beads are oxidized by about 1% to about 5%.

15. The composite of claim 1, wherein the composite comprises about 40 wt % to about 60 wt % cells encapsulated in gel beads.

16. The composite of claim 1, wherein the cells encapsulated in gel beads comprise cells selected from the group consisting of MC3T3 cells, adipose-derived mesenchymal stem cells, marrow-derived mesenchymal stem cells, stem cells, and combinations thereof.

17. The composite of claim 1, wherein the gel beads have an initial mesh size of about 3 nm to about 20 nm.

18. The composite of claim 1, wherein the composite includes blowing-induced pores of about 10 μm to about 150 μm, and wherein at least a portion of the blowing-induced pores are interconnected.

19. The composite of claim 1, wherein the composite has an initial porosity of about 10% to about 50%.

20. The composite of claim 19, wherein the composite has an initial porosity of about 15% to about 40%.

21. The composite of claim 1, wherein the gel beads have a shear modulus of about 2500 Pa to about 250,000 Pa.

22. A method of synthesizing a composite, comprising:
encapsulating cells in gel beads;
mixing the cells in gel beads with at least a prepolymer and a hardener polyol to form a reactive mixture; and
allowing the reactive mixture to react.

23. The method of claim 22, wherein the encapsulating step includes:
mixing cells with an alginate solution to form a cell solution;
adding the cell solution to a gelling agent solution through a nozzle; and
allowing the gel beads to form,
wherein the size of the gel beads is modified by adjusting any of a diameter of the nozzle, adjusting a flow rate the cell solution in the adding step, and adjusting an applied voltage that is applied to the nozzle.

24. The method of claim 23, wherein alginate in the alginate solution includes a partially oxidized alginate.

25. The method of claim 24, wherein the partially oxidized alginate formed by a method including:
reacting a solution including an alginate salt and a sodium periodate;
stopping the reacting step with a reaction inhibitor;
precipitating the solution to collect precipitates; and
redissolving the precipitates.

26. The method of claim 25, wherein the reaction inhibitor is ethylene glycol.

27. The method of claim 23, wherein the gelling agent solution includes CaCl₂, a formulation for culturing cells, water, or combinations thereof.

28. The method of claim 27, wherein the formulation for culturing cells is selected from the group consisting of α-MEM, dionized water, PBS, DMEM, and combinations thereof.

29. The method of claim 22, wherein the hardener polyol includes polyester triol and a catalyst.

30. The method of claim 22, wherein the prepolymer is a lysine triisocyanate-polyethylene glycol prepolymer.

31. The method of claim 22, wherein the cells in gel beads comprise about 40 wt % to about 60 wt % of the composite.

32. The method of claim 23, wherein the alginate solution comprises about 1% to about 2% (w/v) of alginate.

33. The method of claim 23, wherein the gelling agent solution comprises about 100 mM to about 200 mM CaCl₂.

34. The method of claim 22, wherein the cells are selected from the group consisting of MC3T3 cells, adipose-derived mesenchymal stem cells, marrow-derived mesenchymal stem cells, stem cells, and combinations thereof.

35. The method of claim 22, wherein an initial mesh size of the gel beads is about 3 nm to about 20 nm.

36. The method of claim 22, wherein allowing the reactive mixture to react forms the composite having blowing-induced pores in the composite having a size of about 10 μm to about 150 μm, and wherein at least a portion of the blowing-induced pores are interconnected.

37. The method of claim 22, wherein the allowing the reactive mixture to react forms the composite having an initial porosity of about 10% to about 50%.

38. A method of delivering cells to tissue, comprising:
administering to a subject in need thereof an effective amount of a biodegradable composite including a polyurethane component and encapsulated cells.

39. The method of claim 38, wherein administering the biodegradable composite regenerates the tissue.

40. The method of claim 38, wherein the administering an effective amount of the biodegradable composite includes:
injecting or applying the biodegradable composite on the tissue; and
allowing the biodegradable composite to cure on the tissue.

41. The method of claim 38, wherein the tissue is bone tissue, dermal tissue, organ tissue, epithelial tissue, or combinations thereof.

42. The method of claim 38, wherein the encapsulated cells include cells encapsulated in alginate beads.

43. The method of claim 38, wherein the biodegradable composite includes pores having a size of about 50 μm to about 2 mm.

44. The method of claim 38, wherein the encapsulated cells have a size of about 200 μm to about 2 mm.

45. The method of claim 38, wherein the biodegradable composite includes about 40 wt % to about 60 wt % of the encapsulated cells.

46. The method of claim 38, wherein the encapsulated cells are selected from the group consisting of MC3T3 cells, adipose-derived mesenchymal stem cells, marrow-derived mesenchymal stem cells, stem cells, and combinations thereof.

47. The method of claim 42, wherein the alginate beads include partially oxidized alginate.

48. The method of claim 42, wherein the alginate beads further comprise a formulation for cell culture.

49. The method of claim 42, wherein the formulation for cell culture is selected from the group consisting of α-MEM, deionized water, PBS, DMEM, and combinations thereof.
